(19) United States
(12) Patent Application Publication
(10) Pub. No.: US 2007/0219161 A1
(43) Pub. Date: Sep. 20, 2007

(54) Preparation Process Of Oligoglycosaminoglycan, And Reducing End Glucuronic Acid Type Oligochondroitin Sulfate And Pharmaceutical Composition Comprising The Same

(76) Inventors: Junichi Tamura, Tottori (JP); Taro Matsumoto, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/594,447

(22) PCT Filed: Mar. 25, 2005
(86) PCT No.: PCT/JP05/06439

§ 371 Date: Mar. 6, 2007

(30) Foreign Application Priority Data
Mar. 26, 2004 (JP) 2004-093219

(51) Int. Cl. C07H 5/04

(52) U.S. Cl. 514/62; 536/55.3

(57) ABSTRACT

A sugar donor having a glucuronic acid or iduronic acid derivative at the reducing end and in which a leaving group is added to the reducing end hydroxyl group to be glycosylated and the other hydroxyl groups and the carboxyl groups are protected, is subjected to glycosylation reaction with a sugar acceptor having a N-acylgalactosamine derivative at the reducing end and in which the non-reducing end hydroxyl group to be glycosylated is free and the other hydroxyl groups are protected, in the presence of (A-3) a particular promoter. Provided be can a process for preparing an oligoglycosaminoglycon of an intended chain length composed of four or more constituent sugars in highly stereoselectively, high yield and high purity.

### TECHNICAL FIELD

The present invention relates to a chemical preparation process for efficiently preparing an oligoglycosaminoglycan composed of four or more, particularly five or more constituent sugars. The present invention also relates to a reducing end glucuronic acid type chondroitin sulfate oligosaccharide composed of five or more constituent sugars and a pharmaceutical composition containing the same which are useful for improving, treating and preventing a condition or diseases induced by participation of a CD44 molecule.

### BACKGROUND ART

A glycosaminoglycan is a polysaccharide having a repeating structure of a basic disaccharide unit composed of an uronic acid or a galactose or derivative thereof, and a hexosamine or derivative thereof. A glycosaminoglycan in vivo exists as a very long sugar chain formed by repetition of about 40 to 100 times of a basic disaccharide unit, and in most cases covalently binds to a core protein in a proteoglycan.

The followings have been known as a glycosaminoglycan: a chondroitin sulfate, a dermatan sulfate, a heparan sulfate, a heparin, a keratan sulfate, etc. In a chondroitin sulfate, a glucuronic acid or a derivative thereof and a N-acylgalactosamine or a derivative thereof constitute a basic disaccharide unit. In a dermatan sulfate, an iduronic acid or a derivative thereof and a N-acylgalactosamine a derivative thereof constitute it. In keratan sulfate, a galactose or a derivative thereof and a N-acylgalactosamine or a derivative thereof constitute it.

In recent years, a cell recognition function of these glycosaminoglycans has drawn high attention and it has been revealed that a sugar chain containing a glycosaminoglycan expressed in various cells participates in various physiological functions through an interaction with an extracellular component.

For example, JP 2003-512807A discloses that a dermatan sulfate of 16 to 100 sugar units composed of a repeating unit of a disaccharide containing an iduronic acid and a sulfated acetylgalactosamine is useful as an inhibitor of thrombin generation and complement activation. It is also disclosed in ARTHRITIS & RHEUMATISM, Vol. 42, No. 4, 1999, pp. 659-668 that, in a mouse articular rheumatism model, a hyaluronic acid in vivo interacts with a CD44 antigen which is known to participate in various cell functions, and that if the binding of a hyaluronic acid to a CD44 is inhibited by a particular antibody, the condition of articular rheumatism is alleviated.

Furthermore, in the latest researches, it has been suggested or pointed out that an oligoglycosaminoglycan, wherein the term “oligo” herein means that it is composed of 2 to 20 constituent sugars, which can be obtained by cleaving a long-chain glycosaminoglycan extracted from a living body interacts with an extracellular component and participates in a physiological function.

For example, it is disclosed in THE JOURNAL OF BIOLOGICAL CHEMISTRY Vol. 277, No. 15, pp. 12921-12930, 2002 that a chondroitin sulfate E composed of four constituent sugars interacts with L-secretin and P-secretin. However,-this reference also states that a corresponding chondroitin family which is partly or not at all sulfated does not interact with L-secretin and P-secretin. Furthermore, this reference also discloses that a glycosaminoglycan composed of two constituent sugars interacts with a CD44, and more specifically has pointed out that a chondroitin, dermatan, and hyaluronic acid composed of two constituent sugars interact with a CD44 regardless of sulfation or non-sulfation: i.e., a sulfate group on the sugar chain does not contribute to the interaction with a CD44.

This reference describes an analysis of the interaction of each oligoglycosaminoglycan and a CD44 by a plasmon resonance assay, but does not demonstrate any actual physiological functions.

In this regard, it is disclosed in WO 96/16973 that an oligokeratan sulfate composed of 2 to 5 constituent sugars having a sulfated acetylglucosamine at the reducing end is useful as an anti-inflammation agent, an anti-allergy agent, an immunomodulator, a cell differentiation inducing agent, and an apotosis inducing agent.

Moreover, JP 5-178876A describes that an oligochondroitin composed of 2 to 8 constituent sugars which has a basic disaccharide unit composed of a D-galactosamine derivative and a D-glucuronic acid derivative has an anti-allergy effect, an anti-inflammation effect and a hyaluronidase inhibiting effect.

However, there is no description in this publication that an oligosaccharide composed of four or more sugars was actually prepared, as being apparent from the disclosed preparation process wherein a sugar donor composed of monosaccharide was successively bound through a glycosidic linkage. In this reference, of course, a pharmacological activity has been demonstrated only for a chondroitin composed of two constituent sugars.

It is indicated in THE JOURNAL OF BIOLOGICAL CHEMISTRY Vol. 278, No. 34, pp. 32259-32265, 2003 that an oligomer mixture containing several oligohyaluronic acids of different chain lengths composed of 6 to 14 constituent sugars induces the degradation of a CD44, whereas a hyaluronic acid composed of 1000 or more constituent sugars as well as a hyaluronic acid composed of two constituent sugars do not induce a CD44 degradation.

Meanwhile, the demand for a process of selectively preparing an oligoglycosaminoglycan having a particular chain. length, a modifying group formed by adding a particular group at the particular position, for example, by sulfation and a particular stereo structure is becoming strong, as the function of oligoglycosaminoglycan increasingly draws attention.

Conventionally, as one process for obtaining an oligoglycosaminoglycan, a process of cleaving a long chain glycosaminoglycan extracted from a living body by using a cleaving enzyme is known: for example have been disclosed in WO 96/16973 and JP 5-058716A.

However, this enzyme process tends to come into question in the side effects due to contamination of other components in the living body, when applied to preparation of a medicine. In addition, according to this enzymatic process, a glycosaminoglycan of an intended chain length cannot be obtained and what was actually obtained was mostly that composed of two constituent sugars. In addition, there is also the limitation that the modifying group and stereo structure are fundamentally determined by the glycosaminoglycan extracted from in a living body.

On the other hand, as a process of obtaining a glycosaminoglycan which has no contamination by impurities and has an intended chain length, a group modified at an intended position, and/or an intended stereo structure, a chemical synthetic process attracts attention.

For example, the above-mentioned JP-5-178876-A discloses a process wherein a D-galactosamine derivative and a D-glucuronic acid derivative are successively bound with each other through a glycosidic linkage to prepare a 2- to 8-oligosaccharide composed of repetition of a basic disaccharide unit thereof.

However, even though this process requires the steps of protecting it with a protecting group and eliminating the protecting group every time for connecting a monosaccharide one by one, no consideration is made for obtaining a glycosaminoglycan composed of four or more constituent sugars in high yield. Actually, this reference does not describe any example demonstrating a preparation of a glycosaminoglycan composed of four or more constituent sugars. This reference does not also describe any processes of sulfating selectively the hydroxyl group at a particular position.

On the other hand, the present inventors have reported a process for obtaining a reducing end glucuronic acid type tetrasaccharide chondroitin sulfate by reacting a sugar donor composed of two azidized constituent sugars with a sugar acceptor composed of two similarly azidized constituent sugars in the presence of BF₃.OEt₂ which is a Lewis acid (Carbohydrate Research 305 (1998) 43-63 and Bioorganic & Medicinal Chemistry Letters, Vol. 5, No. 13, pp. 1351-1354, 1995).

This process solves the problem in the process disclosed in the above-mentioned patent publication and enables a reducing end glucuronic acid type tetrasaccharide chondroitin sulfate to be obtained in a yield of 50%.

According to the preparation process described in these references, however, it was difficult to synthesize a reducing end glucuronic acid type chondroitin sulfate composed of five or more constituent sugars, because the azide group was not able to be converted into an acetamide group when an acetylgalactosamine composed of five or more constituent sugars is synthesized.

Carbohydrate Research 326 (2000), 88-97 discloses a process of selectively sulfating an intended hydroxyl group of a glycosaminoglycan. However, the process of this reference was a process of sulfating a N-acylgalactosamine at either the 4- or 6-position, but was not a process which can selectively sulfate that at both the 4- or 6- positions.

### DISCLOSURE OF THE INVENTION

The present invention has been made in view of the above-mentioned problems of the prior art, and aims at providing a process of preparing highly stereoselectively an oligoglycosaminoglycan of an intended chain length and a structure composed of four or more, particularly five or more constituent sugars in high yield and high purity. In addition, the present invention aims at providing a high pure of reducing end glucuronic acid type oligochondroitin sulfate composed of five or more intended number of constituent sugars obtained for the first time by the preparation process of the present invention and a pharmaceutical composition containing the same.

For solving the above-mentioned problems, the present inventors have reviewed the preparation process described in the above-mentioned Carbohydrate Research 305 (1998), 43-63. The inventors have then found that an oligoglycosaminoglycan of an intended chain length composed of four or more constituent sugars can be prepared in high yield and high purity by using a sugar donor and sugar acceptor composed of an acetamidated constituent sugar and also using as a promoter, a Lewis acid which is a counter ion for the sugar donor, for example, trimethylsilyl trifluoromethanesulfonate or an analogue compound thereof. Thus, this finding has led to the accomplishment of the present invention.

That is, the present invention provides a process of preparing an oligoglycosaminoglycan or its characteristic intermediate obtained in an unique glycosylation reaction step thereof wherein the process is characterized in comprising a step(A) of subjecting a sugar donor which has a glucuronic acid or iduronic acid derivative at the reducing end and in which a leaving group is added to the reducing end hydroxyl group to be glycosylated and the other hydroxyl groups and the carboxyl groups are protected, to glycosylation reaction with a sugar acceptor which has a N-acylgalactosamine derivative at the non-reducing end and in which the non-reducing end hydroxyl group to be glycosylated is free and the other hydroxyl groups are protected, in the presence of a Lewis acid as a promoter which is present as a counter ion for the sugar donor, for example, a compound represented by the general formula (1):

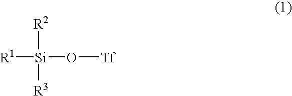

In one preferred embodiment, the present invention also provides a process of preparing an oligoglycosaminoglycan or an intermediate thereof wherein the process is characterized in comprising a step(A) of subjecting a sugar donor containing a glucuronic acid or iduronic acid derivative in which a leaving group is added to the reducing end hydroxyl group to be glycosylated and the other hydroxyl groups and the carboxyl groups are protected, or an oligosaccharide derivative, usually composed of two to ten constituent sugars, containing as a basic constituent unit a basic disaccharide unit composed of a N-acylgalactosamine derivative, and a glucuronic acid or iduronic acid derivative in which a leaving group is added to the reducing end hydroxyl group to be glycosylated and the other hydroxyl groups and the carboxyl groups are protected, to a glycosylation reaction with a sugar acceptor containing a glucuronic acid or iduronic acid derivative at the non-reducing end in which the non-reducing end hydroxyl group to be glycosylated is free and the other hydroxyl groups and the carboxyl groups are protected, or an oligosaccharide derivative including as a basic constituent unit a basic disaccharide unit composed of a N-acylgalactosamine derivative, and a glucuronic acid or iduronic acid derivative. in which the non-reducing end hydroxyl group to be glycosylated is free and the other hydroxyl groups and the carboxyl groups are protected; in the presence of the above-mentioned promoter.

In one preferred embodiment, the preparation process of the present invention further comprises, in addition to the. above-mentioned step (A), a step (B) of eliminating one protecting group at the non-reducing end of the oligosaccharide derivative obtained in the above step (A); and a step (C) of subjecting the oligosaccharide derivative from which the one protecting group is eliminated to a glycosylation reaction with the above-mentioned sugar acceptor, preferably composed of one or two constituent sugars, in the presence of the above-mentioned promoter. An oligoglycosaminoglycan with an intended chain length composed of five or more constituent sugars can be prepared by repeating the steps (B) and (C) in a predetermined number of times within 1 to 8. It is desirable, in respect of a high yield, to prepare an oligoglycosaminoglycan with an intended chain length by repeating the steps (B) and (C) in 1 to 5 times.

In the preparation process of the present invention, the above-mentioned sugar donor and sugar acceptor may be selected depending on the target oligoglycosaminoglycan. For example, when preparing a reducing end glucuronic acid type oligoglycosaminoglycan or a derivative thereof, the following can be used as a sugar donor: a glucuronic acid or iduronic acid derivative in which a leaving group is added to the reducing end hydroxyl group to be glycosylated and the other hydroxyl groups and the carboxyl groups are protected; or an oligosaccharide derivative, usually composed of two to ten constituent sugars, containing as a basic constituent unit a basic disaccharide unit composed of a N-acylgalactosamine derivative in which all the hydroxyl groups are protected and a glucuronic acid or iduronic acid derivative in which a leaving group is added to the reducing end hydroxyl group to be glycosylated and the other hydroxyl groups and the carboxyl groups are protected. More specifically, when preparing an oligochondroitin or a derivative thereof, the following can be used: a sugar donor composed of one or two constituent sugars represented by the general formula (2) or general formula (2′):

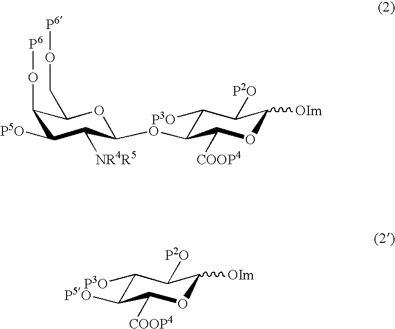

Similarly, when preparing a reducing end glucuronic acid type oligoglycosaminoglycan or a derivative thereof, the following can be used as a sugar acceptor: a glucuronic acid or iduronic acid derivative in which the non-reducing end hydroxyl group to be glycosylated is free and the other hydroxyl groups and the carboxyl groups are protected; or an oligosaccharide derivative, usually composed of two to ten constituent sugars, containing as a basic constituent unit a basic disaccharide unit composed of a N-acylgalactosamine derivative in which the non-reducing end hydroxyl group to be glycosylated is free and the other hydroxyl groups and the carboxyl groups are protected and a glucuronic acid or iduronic acid derivative in which all the hydroxyl groups and the carboxyl groups are protected. More specifically, in a preferred embodiment of preparing an oligochondroitin or derivative thereof, the sugar acceptor composed of two constituent sugars represented by the general formula (3) can be used:

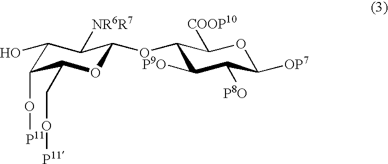

The promoter used in the present invention is preferably a compound represented by in the above-mentioned general formula (1) in which R¹, R², and R³ are respectively the same or independently a hydrogen atom, or a linear or branched alkyl group. Particularly, preferable is a compound in which the alkyl group has five or less carbon atoms such as trimethylsilyl trifluoromethanesulfonate (TMSOTf).

The preparation process of the present invention generally comprises a step of eliminating all the protecting groups of the oligosaccharide obtained in the above step (A) or (C). The present process may comprise, along with a step of eliminating all the protecting groups of the oligosaccharide obtained in the above step (A) or (C), a step of selectively sulfating each N-acetylglucosamine at the 4th and/or 6th position(s) depending on the purpose. Needless to say, the eliminating step of these protecting groups and/or the sulfating step can be conducted by another person separated from the above step (A) or (C).

In one preferable embodiment, the present invention may comprise the steps of the hydroxyl groups at the 4th and 6th positions in each N-acylgalactosamine of the oligosaccharide obtained in the above step (A) or (C) being protected with benzylidene, alkoxybenzylidene and/or cyclohexylidene; the hydroxyl groups at a position other than the 4th and 6th positions in the constituent sugar of the non-reducing end oligosaccharide being protected with a pivaloyl group; the benzylidene, alkoxybenzylidene and/or cyclohexylidene which were made to protect the hydroxyl groups at the 4th and 6th positions being eliminated; and subsequently the deprotected hydroxyl groups at the 4th and 6th positions being sulfated to selectively sulfate the 4th and 6th positions in N-acylgalactosamine.

The present invention also encompasses the intermediate of an oligoglycosaminoglycan having a characteristic structure which are generated in the steps of the preparation process of the present invention, as represented by the general formula (4′):

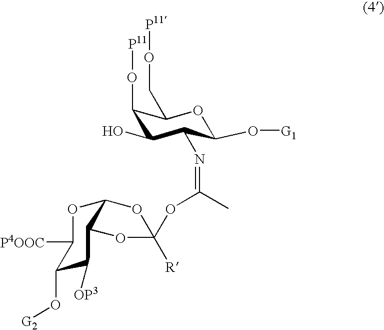

The present invention further provides a novel reducing end glucuronic acid type. oligochondroitin, a reducing end glucuronic acid type oligochondroitin sulfate or a reducing end iduronic acid type oligochondroitin sulfate, or a salt or a derivative thereof: hereinafter sometimes collectively referred to “a (the) reducing end glucuronic acid type oligochondroitin or a (the) sulfate thereof etc.” Specifically, the present invention provides a reducing end glucuronic acid type oligochondroitin or a (the) sulfate thereof etc. represented by the general formula (4):

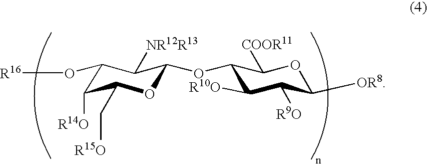

In the reducing end glucuronic acid type oligochondroitin or the sulfate thereof etc. of the present invention, in terms of the shedding inducing ability to a CD44, preferable is a compound represented by the below-mentioned general formula (4) in which at least one of R¹⁴ and R¹⁵ is a sulfate group optionally substituted with any one selected from the group consisting of sodium, potassium, copper, calcium, iron, manganese, zinc, ammonium, barium and lithium, and particularly preferable is a compound represented by the formula (4) in which both of R¹⁴ and R¹⁵ is a sulfate group optionally substituted with any one selected from the group consisting of sodium, potassium, copper, calcium, iron, manganese, zinc, ammonium, barium and lithium. As for the reducing end glucuronic acid type oligochondroitin or the sulfate thereof etc. of the present invention, in term of efficient production and the shedding inducing ability to a CD44, preferable is a compound represented by the below-mentioned general formula (4) in which n is 3 to 6.

The reducing end glucuronic acid type oligochondroitin or the sulfate thereof etc. of the present invention does not at all contain impurities such as a lipid and protein which are other living body components.

The reducing end glucuronic acid type oligochondroitin or the sulfate thereof etc. of the present invention has high activity in the shedding inducing ability to a CD44, and can be thus used as an active ingredient for improving, treating or preventing a disease or condition induced by a CD44 molecule.

In this way, the present invention also provides a pharmaceutical composition for improving, treating or preventing a disease or condition induced by a CD44 molecule containing the reducing end glucuronic acid type oligochondroitin or the sulfate thereof etc. of the present invention and a pharmacologically acceptable carrier.

Moreover, the present invention also provides use of the reducing end glucuronic acid type oligochondroitin or the sulfate thereof etc. of the present invention for preparing a pharmaceutical composition which improves, treats or prevents a disease or condition induced by the action of a CD44 molecule.

Furthermore, the present invention provides a process of improving, treating or preventing a disease or condition induced by the action of a CD44 molecule which comprises administering to a subject the reducing end glucuronic acid type oligochondroitin or the sulfate thereof etc. of the present invention

### BRIEF DESCRIPTION OF THE DRAWINGS

FIG. 1 is a reaction scheme showing the outline of the step (A) in one embodiment of the preparation process of the present invention.

FIG. 2 is a reaction scheme showing the outline of the step (B) in one embodiment of the preparation process of the present invention.

FIG. 3 is a reaction scheme showing the outline of the step (C) in one embodiment of the preparation process of the present invention.

FIG. 4 is a reaction scheme showing the outline of the protecting group eliminating step in one embodiment of the preparation process of the present invention.

FIG. 5 is a reaction scheme showing the outline of the selective sulfation step in one embodiment of the preparation process of the present invention.

FIGS. 6-1 and 6-2 are reaction schemes showing the outline of the steps in the preparation process of Example 1.

FIG. 6-3 is a reaction scheme showing the outline of the preparation process of Examples 2 and 3.

FIG. 7 is a reaction scheme showing the outline of the preparation process of Comparative Example 1.

FIG. 8 is a flowchart showing the outline of the test method performed in Example 4.

FIG. 9 is a copy of the electrophoresis photograph in which the electrophoresis result of the test performed in Example 4 is shown.

FIG. 10 is a graph which shows the CD44 shedding index in the test performed in Example 4. the CD44 shedding index was calculated assuming that the state without stimulation is 1;

FIG. 11 is a reaction scheme showing another embodiment of the preparation process of the present invention.

FIG. 12 is a reaction scheme showing the outline of the reaction mechanism in one embodiment of the present invention.

### BEST MODE FOR CARRYING OUT THE INVENTION

The preparation process of the present invention is a process comprising a step (A) of subjecting a sugar donor having at the end a glucuronic acid or iduronic acid derivative in which a leaving group is added to the reducing end hydroxyl group to be glycosylated and the other hydroxyl groups and the carboxyl groups are protected; to glycosylation reaction with a sugar acceptor having at the end a N-acylgalactosamine derivative in which the non-reducing end hydroxyl group to be glycosylated is free and the other hydroxyl groups are protected; in the presence of a particular Lewis acid type promoter.

In one preferred embodiment, the preparation process of the present invention, further comprises, the step (B) of eliminating a protecting group at the non-reducing end of the oligosaccharide obtained in the above step (A); and the step (C) of subjecting the oligosaccharide having the free hydroxyl group at the non-reducing end to a glycosylation reaction with the above-mentioned sugar acceptor in the presence of the above-mentioned promoter, and repeats the steps (B) and (C) in an intended number of times within 1 to 8.

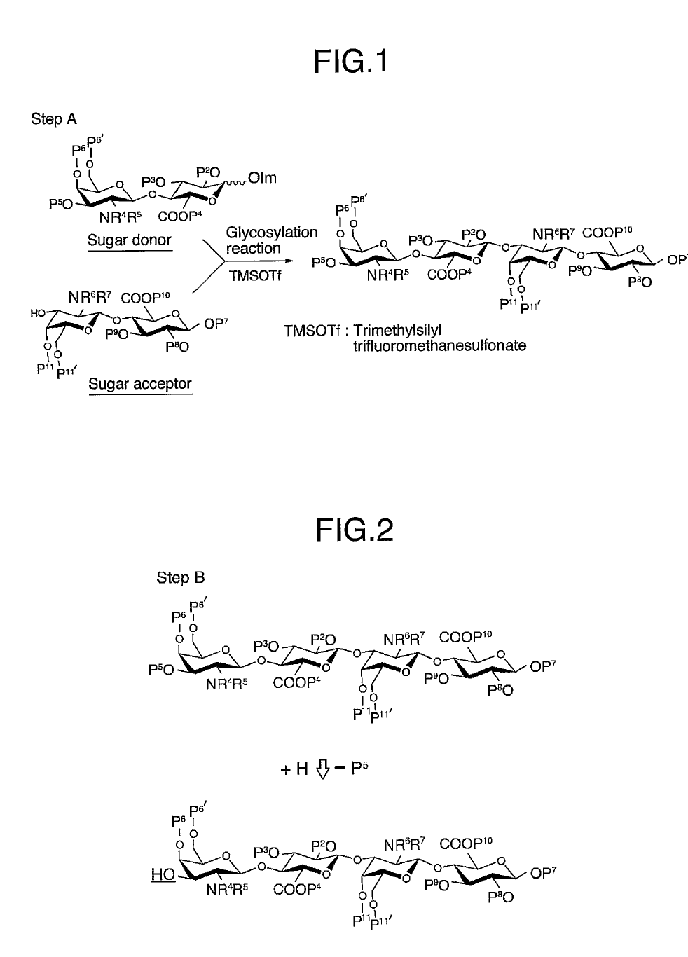

Hereinafter, each step is described specifically referring to FIGS. 1 to 5 and 11 showing the reaction scheme in one embodiment of the present invention. FIGS. 1 to 5 and 11 show the reaction scheme in a reaction for obtaining a reducing end glucuronic acid type chondroitin sulfate composed of 5 or 6 constituent sugars, as mentioned later as a typical example. Therefore, the present invention should not be restricted at all by these drawings. Then, it should be understood that, when another oligoglycosaminoglycan is to be prepared, a corresponding sugar donor, sugar acceptor and protecting group are suitably selected depending on the target oligoglycosaminoglycan.

In the present invention as mentioned below, the groups “alkyl”, “alkenyl”, aralkyl”, “alkoxy”, “aryl”, “alkylidene”, “acyl” and “ether” usually contain the following carbon atoms, respectively:
alkyl group: usually 1 to 10, preferably 1 to 7, more preferably 1 to 5;
alkenyl group: usually 2 to 10, preferably 1 to 7, more preferably 1 to 5;
aralkyl group: usually 6 to 30, preferably 7 to 20, more preferably 6 to 15;
alkoxy group: usually 1 to 10, preferably 1 to 7, more preferably 1 to 5;
aryl group: u usually 5 to 20, preferably 6 to 15, more preferably 6 to 13;
alkylidene group: usually 1 to 10, preferably 1 to 7, more preferably 1 to 5;
acyl group: usually 1 to 20, preferably 1 to 10, more preferably 2 to 7; and
ether: usually 2 to 20, preferably 2 to 10, more preferably 2 to 7
   (A) Glycosylation of an acetamidated sugar donor and sugar acceptor
   (A-1) Sugar donor

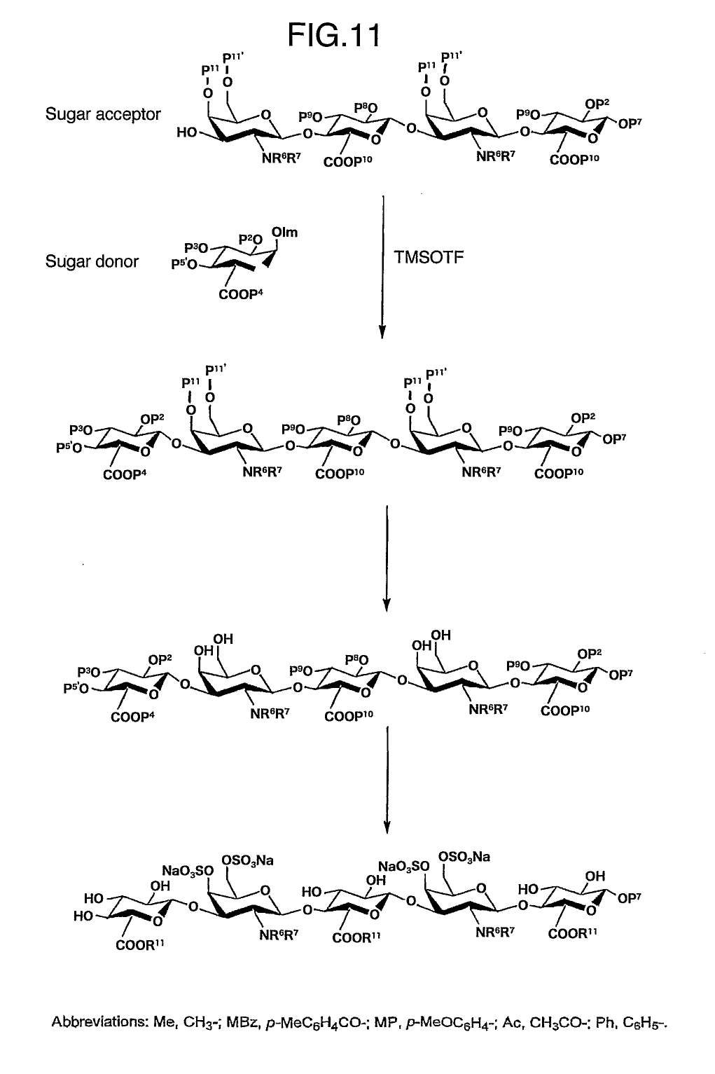

As shown in FIGS. 1 and 11, a sugar donor used in the present invention may be a compound having at the end a glucuronic acid or iduronic acid derivative in which a leaving group, shown as Im in FIGS. 1 and 11, is added to the reducing end hydroxyl group to be glycosylated and the other hydroxyl groups and the carboxyl groups are protected, as shown as P2 to P6 and P6′ in FIGS. 1 and 11. Further, the following is preferable: a glucuronic acid or iduronic acid derivative in which a leaving group, shown as Im in FIG. 11, is added to the reducing end hydroxyl group to be glycosylated and the other hydroxyl groups and the carboxyl groups are protected, as shown as P2 to P5′ in FIG. 11; or an oligosaccharide derivative, usually composed of two to ten constituent sugars, having as a basic constituent unit a basic disaccharide unit composed of a N-acylgalactosamine derivative and a glucuronic acid or iduronic acid derivative in which a leaving group, shown as Im in FIG. 1, is added to the reducing end hydroxyl group to be glycosylated and the other hydroxyl groups and the carboxyl groups are protected, as shown as P2 to P6 and P6′ in FIG. 1.

By using such a sugar donor composed of an acetamidated constituent sugar, the problem can be avoided which is in low yield resulting from the acetamidation when extended to four or more sugars.

As for a sugar donor used in the present invention, there is no limitation in other points and the sugar donor may be selected depending on the type of the target oligoglycosaminoglycan.

For example, when preparing an oligochondroitin sulfate, a sugar donor may contain the following: a glucuronic acid derivative in which the hydroxyl group to be glycosylated in the constituent sugar at the reducing end is imidated and the other hydroxyl groups and the carboxyl groups are protected; or an oligosaccharide derivative containing as a basic constituent unit a basic disaccharide unit composed of a N-acylgalactosamine derivative and a glucuronic acid derivative in which the hydroxyl group to be glycosylated in the constituent sugar at the reducing end is imidated and the other hydroxyl groups and the carboxyl groups are protected.

Similarly, when preparing a dermatan sulfate, a sugar donor may contain the following: an iduronic acid derivative in which the hydroxyl group to be glycosylated in the constituent sugar at the non-reducing end is imidated and the other hydroxyl groups and the carboxyl groups are protected; or an oligosaccharide derivative containing as a basic constituent unit a basic disaccharide unit composed of a N-acylgalactosamine derivative and a iduronic acid derivative in which the hydroxyl group to be glycosylated in the constituent sugar at the non-reducing end is imidated and the other hydroxyl groups and the carboxyl groups are protected. When preparing a keratan sulfate, a sugar donor may contain the following: a galactose derivative in which the hydroxyl group to be glycosylated in the constituent sugar at the non-reducing end is imidated and the other hydroxyl groups and the carboxyl groups are protected; or an oligosaccharide derivative containing as a basic constituent unit a basic disaccharide unit composed of a N-acylgalactosamine derivative and a galactose derivative in which the hydroxyl group to be glycosylated in the constituent sugar at the non-reducing end is imidated and the other hydroxyl groups and the carboxyl groups are protected.

A protecting group of these sugar donors may contain, for example, an alkyl group such as methyl and ethyl; an aralkyl group such as benzyl and methylbenzyl; an alkoxybenzyl group such as p-methoxybenzyl; a triphenylalkyl group such as triphenylmethyl; an alkenyl group such as allyl; a halogen; a thioalkyl group such as thiomethyl; an alkylidene such as isopropylidene; a benzylidene group optionally substituted with an alkyl group or an alkoxy group such as benzylidene and alkoxybenzylidene such as p-methoxybenzylidene; a cyclohexylidene group optionally substituted with an alkyl group or an alkoxy group; an acyl group optionally substituted with a halogen such as benzoyl, acetyl and monochloroacetyl; a sulfonyl group; or a silyl or silyl ether group optionally substituted with an alkyl group or an alkoxy group.

In the present invention, it is desirable to suitably design a protecting group and a substituent group depending on the target compound so that the compound having a desired structure may be obtained by the extension through a glycosylation reaction and the selective addition of a sulfate group, etc.

For example, a protecting group, for example P5 in FIG. 1, at the position to be subjected to a glycosylation reaction with a sugar donor added later is preferably a acetyl group optionally substituted with a halogen, an alkenyl group such as allyl, an acyl group, an aralkyl group or a silyl group optionally substituted with an alkyl group or an alkoxy group such as trimethylsilyl, and particularly preferably monochlroacetyl, p-methoxybenzyl or levulinoyl so that only the protecting group at the desired position can be eliminated in advance of a glycosylation.

In addition, in order to make the reaction go through an intermediate which has a stable carbocation ortho ester structure to obtain a desired final product in high yield, it is preferable to protect the hydroxyl group at the 2nd position of the sugar donor with an electron donation substituent, for example an acyl group, particularly preferably with a benzoyl group optionally substituted with an alkyl group or an alkoxy group in that the conjugate system of ortho ester carbocation is long and a stable cation structure is readily formed: In FIGS. 1 and 11, a protecting group at the 2nd position is shown as P2. The benzoyl group optionally substituted may include benzoyl, methylbenzoyl, ethylbenzoyl, propylbenzoyl, dimethylbenzoyl, methoxylbenzoyl, ethoxylbenzoyl and dimethoxylbenzoyl and the like, and among them methoxylbenzoyl is preferable.

Furthermore, in order to perform selectively sulfation at 4th and 6th positions of each N-acylgalactosamine, these positions are preferably protected with benzylidene, alkyloxybenzylidene such as p-methoxybenzylidene or cyclohexylidene: In FIG. 1, the protecting groups at 4th and 6th positions are shown as P6 and P6′.

Moreover, when protecting a particular position over the preparation process, an alkyl group such as methyl; an aralkyl group such as benzyl and methylbenzyl; an alkylaromatic group such as triphenylmethyl; an alkyloxybenzyl group such as p-methoxybenzyl; an alkenyl group such as allyl; or an acyl group optionally substituted with a halogen such as benzoyl, acetyl and monochloroacetyl is preferably used as a protecting group. These protecting groups may be formed by any method well known in the art.

As preferable examples of a sugar donor used in the present invention, a sugar donors used for preparing a reducing end glucuronic acid type oligochondroitin sulfate as mentioned below are shown in the general formulas (2) and (2′):

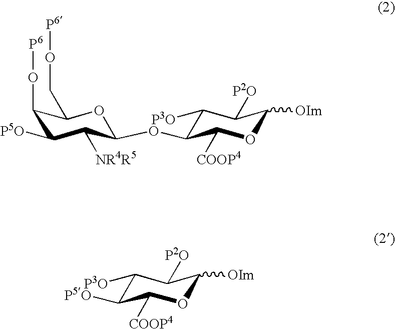

In the general formulas (2) and (2′), R⁴ and R⁵ are the same or independently selected from the group consisting of a hydrogen atom, an acetyl group optionally substituted with a halogen, an alkyl group, an alkenyl group such as allyl, an acyl group optionally substituted with a halogen, and a phthaloyl group, and preferably selected from the group consisting of acetyl, haloacetyl, benzoyl and phthaloyl.

Im is an imidoyl group optionally substituted with a halogen, and preferably a leaving group selected from the group consisting of trichloroacetimidoyl, trifluoroacetimidoyl and acetimidoyl.

P² and P³ are the same or independently selected from the group consisting of a hydrogen atom, an alkyl group, an alkenyl group such as allyl, an aralkyl group, an aryl group and a silyl group optinally substituted with an alkyl group or an alkoxy group such as trimethylsilyl, and preferably selected from the group consisting of benzyl, alkylbenzyl, triphenylalkyl and silyl.

P⁴ is selected from the group consisting of an alkyl group, an alkenyl group such as allyl and an aralkyl group, and preferably selected from the group consisting of benzyl, alkylbenzyl and haloalkyl.

P⁵ and P⁵′ are selected from the group consisting of an benzyl group optionally substituted with an alkyl group or an alkoxy group, an alkenyl group such as allyl, an acyl group optionally substituted with a halogen such as acetyl optionally substituted with a halogen, an aralkyl group and a silyl group optionally substituted with an alkyl group or an alkoxy group such as trimethylsilyl, and preferably selected from the group consisting of monochloroacetyl, p-methoxybenzyl and levulinoyl.

P⁶ and P⁶′ are the same or independently selected from the group consisting of a hydrogen atom, an alkyl group, an alkenyl group such as allyl, an aralkyl group, an aryl group, a silyl group optionally substituted with an alkyl group or an alkoxy group such as trimethylsilyl and an alkylidene group, and preferably selected from the group consisting of benzyl, benzylidene and silyl.

Specific examples of the above-mentioned sugar donor may be methyl-(2-acetamide-4,6-O-benzylidene-2-deoxy-3-O-levulinoyl-β-D-galactopyranosyl)-(1->4)-2,3-di-O-(4-methylbenzoyl)-1-O-trichloroacetimidoyl-α-D-glucopyranuronate) and methyl-2,3-di-O-(4-methylbenzoyl)-1-O-trichloroacetimidoyl-α-D-glucopyranuronate).

These sugar donors can be obtained according to a conventionally known method. For example, these sugar donors can be obtained according to a method described in Carbohydrate Research 305 (1998) 43-63, which is incorporated herein by reference.

(A-2) Sugar acceptor

As shown in FIGS. 1 and 11, a sugar acceptor used in the present invention may be a compound having at the end a N-acylgalactosamine derivative, or a glucuronic derivative acid or a iduronic acid derivative when it is composed of one constituent sugar, in which the non-reducing end hydroxyl group to be glycosylated is free and the other hydroxyl groups are protected. Preferably, such a sugar acceptor is the following: an oligosaccharide derivative, usually composed of two to ten constituent sugars, containing as a basic constituent unit a basic disaccharide unit composed of a N-acylgalactosamine derivative and a glucuronic acid or iduronic acid derivative in which the hydroxyl group to be glycosylated in the constituent sugar at the non-reducing end is free, which corresponds to the 3rd position hydroxyl group of the N-acylgalactosamine derivative in FIG. 1, and the other hydroxyl groups and the carboxyl groups are protected, as shown as P7 to P11 and P11′ in FIGS. 1 and 11; or a glucuronic acid or iduronic acid derivative in which the non-reducing end hydroxyl group to be glycosylated is free and the other hydroxyl groups and the carboxyl groups are protected, not shown on any FIGS. 1 and 11.

Similarly as the sugar donor, the problems of low yield resulting from the acetamidation when extended to four or more sugars can be avoided by using a sugar acceptor composed of an acetamidated constituent sugar.

The sugar acceptor is not limited in any other points and may be selected depending on the type of the target oligoglycosaminoglycan.

For example, when preparing an oligochondroitin sulfate, a sugar acceptor may be the following: a glucuronic acid derivative in which the hydroxyl group to be glycosylated in the constituent sugar at the non-reducing end is free and the other hydroxyl groups and the carboxyl groups are protected; or an oligosaccharide derivative containing as a basic constituent unit a basic disaccharide unit composed of a N-acylgalactosamine derivative and a glucuronic acid derivative in which the hydroxyl group to be glycosylated in the constituent sugar at the non-reducing end is free and the other hydroxyl groups and the carboxyl groups are protected.

Similarly, when preparing a dermatan sulfate, a sugar acceptor may be the following: a iduronic acid derivative in which the hydroxyl group to be glycosylated in the constituent sugar at the non-reducing end is free and the other hydroxyl groups and the carboxyl groups are protected; or an oligosaccharide derivative containing as a basic constituent unit a basic disaccharide unit composed of a N-acylgalactosamine derivative and a iduronic acid derivative in which the hydroxyl group to be glycosylated in the constituent sugar at the non-reducing end is free and the other hydroxyl groups and the carboxyl groups are protected. When preparing a keratan sulfate, a-sugar acceptor may be the following: a galactose derivative in which the hydroxyl group to be glycosylated in the constituent sugar at the non-reducing end is free and the other hydroxyl groups and the carboxyl groups are protected; or an oligosaccharide derivative containing as a basic constituent unit a basic disaccharide unit composed of a N-acylgalactosamine derivative and a galactose derivative in which the hydroxyl group to be glycosylated in the constituent sugar at the non-reducing end is free and the other hydroxyl groups and the carboxyl groups are protected.

A protecting group of these sugar acceptors may include, for example, an alkyl group such as methyl and ethyl; an aralkyl group such as benzyl; a triphenylalkyl group such as triphenylmethyl; an alkenyl group such as allyl; a halogen; a thioalkyl group such as thiomethyl; an alkylidene group such as isopropylidene; a benzylidene group optionally substituted with an alkyl group or an alkoxy group such as benzylidene and alkoxybenzylidene such as a p-methoxybenzylidene; a cyclohexylidene group optionally substituted with an alkyl group or an alkoxy group; an acyl group optionally substituted with a halogen such as benzoyl and acetyl optionally substituted with a halogen, for example acetyl and monochloroacetyl; a sulfonyl group; a silyl group optionally substituted with an alkyl group or an alkoxy group such as silyl ether; or an alkenyl group.

In the present invention, it is desirable to suitably design a protecting group and a substituent group for the sugar acceptor depending on the target compound in the similar manner as for the sugar donor.

For example, the hydroxyl group at the position of an anomeric carbon in the constituent sugar at the reducing end is preferably protected with an alkoxyaromatic group such as p-methoxyphenyl: In FIGS. 1 and 11, the protecting group for such a hydroxyl group is shown as P7.

Similarly, for example, in order to perform selectively the sulfation at 4th and 6th positions of each N-acylgalactosamine, these positions are preferably protected with benzylidene, alkyloxybenzylidene such as p-methoxybenzylidene, or cyclohexylidene: In FIGS. 1 and 11, the protecting group at 4th and 6th positions are shown as P11 and P11′.

Moreover, when protecting a particular position over the preparation process, an alkyl group such as methyl, an aralkyl group such as benzyl and methylbenzyl, an alkylaromatic group such as triphenylmethyl, an alkyloxybenzyl group such as p-methoxybenzyl, an alkenyl group such as allyl, a acyl group such as benzoyl or acetyl optinally substituted with a halgen, for example acetyl or monochloroacetyl, is preferably used as a protecting group. These protecting groups may be formed by any method well known in the art.

Here, as a preferable embodiment of a sugar acceptor used in the present invention, a sugar acceptor used for preparing a reducing end glucuronic acid type oligochondroitin sulfate as mentioned below are shown in the general formula (3):

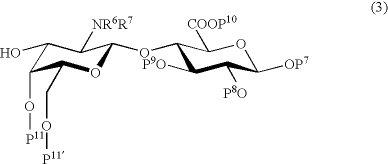

In the general formula (3), R⁶ and R⁷ are the same or independently selected from the group consisting of a hydrogen atom, an alkyl group, an alkenyl group such as allyl, an acyl group optionally substituted with a halogen such as acetyl optionally substituted with a halogen, and a phthaloyl group, and preferably selected from the group consisting of acetyl, haloacetyl, benzoyl and phthaloyl.

P7 is selected from the group consisting of an alkyl group, an aralkyl group, an alkenyl group such as allyl and an aryl group, and preferably selected from the group consisting of phenyl, alkylphenyl, alkoxylphenyl, benzyl, alkylbenzyl, alkoxybenzyl, naphthyl and triphenylalkyl.

P⁸ and P⁹ are the same or independently selected from the group consisting of a hydrogen atom, an alkyl group, an alkenyl group such as allyl, an aralkyl group, an aryl group, an acyl group and a silyl group optionally susbstituted by an alkyl group or an alkoxy group such as trimethylsilyl, and preferably selected from the group consisting of benzyl, alkylbenzyl, triphenylalkyl and silyl.

P¹⁰ is selected from the group consisting of an alkyl group optionally substituted with a halgen, an alkenyl group such as allyl and an aralkyl group, and preferably selected from the group consisting of benzyl, alkylbenzyl and halolalkyl.

P¹¹ and P^{11′} are the same or independently selected from the group consisting of a hydrogen atom, an alkyl group, an alkenyl group such as allyl, an aralkyl group, an aryl group, a silyl group optionally substituted with an alkyl group or an alkoxy group, an alkenyl group such as trimethylsilyl and an alkylidene group, and preferably selected from the group consisting of benzyl, benzylidene and silyl, which include a protecting group in which both are cross-linked to each other.

Specific examples of the above-mentioned sugar acceptor may be methyl-(2-acetamide-4,6-O-benzylidene-2-deoxy-β-D-galactopyranosyl)-(1->4)-[4-methoxyphenyl-2,3-di-O-(4-methylbenzoyl)-α-D-glucopyranoside]uronate.

These sugar acceptors can be obtained according to a conventionally known method. For example, these sugar acceptor can be obtained according to a method described in Carbohydrate Research 305 (1998) 43-63 or Bioorganic & Medicinal Chemistry Letters, Vol. 5, No. 13, pp. 1351-1354, 1995 as mentioned above, which are incorporated herein by reference.

(A-3) Promoter

As shown in FIGS. 1 and 11, in the present invention, the above-mentioned sugar donor is subjected to a glycosylation reaction with the above-mentioned sugar acceptor by means of a Lewis acid having a counter ion which is used as a promoter that can active the leaving group of the sugar donor.

In the present invention, particularly preferably used is a promoter represented by the following general formula (1):

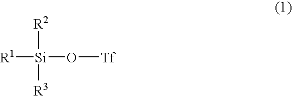

In the above general formula (1), R¹, R² and R³ are the same or independently represent a hydrogen atom, or a linear or branched alkyl group or an aromatic group which is unsubstituted or of which at least some hydrogen atoms are substituted, and Tf represents a trifluoromethanesulfonyl group.

By using the above-mentioned promoter, a yield can be raised surprisingly 20% or more, compared with a ordinary method, namely a method using BF₃-OEt₂.

Further the present method can synthesize a chondroitine type sugar chain composed of 5 or more constituent sugar, which is an acetamide type sugar chain, or a sugar chain which is possible of being converted to the chondroitine type sugar chain, which could not be synthesized by an ordinary method.

The above-mentioned promoters may include trimethylsilyl trifluoromethanesulfonate, triethylsilyl trifluoromethanesulfonate, tripropylsilyl trifluoromethanesulfonate, dimethylethylsilyl trifluoromethanesulfonate, tribenzylsilyl trifluoromethanesulfonate, trinaphthylsilyl trifluoromethanesulfonate or tribenzylmethylsilyl trifluoromethanesulfonate.

In the present invention, a promoter represented by the above-mentioned general formula (1) in which R¹, R² and R³ are a hydorogen atom, or a linear or branched alkyl group is particularly preferable in terms of a yield. Usually, trimethylsilyl trifluoromethanesulfonate (TMSOTf) is used since it is easily available.

The glycosylation reaction in the present invention is usually performed at a temperature of −40 to 40° C. for 12 to 48 hours. In addition, it is preferable to remove for example water and a hydracid halide in the system with a capturing material such as a molecular sieve.

(B) Elimination of the protecting group at a position to be glycosylated in the constituent sugar at the reducing end

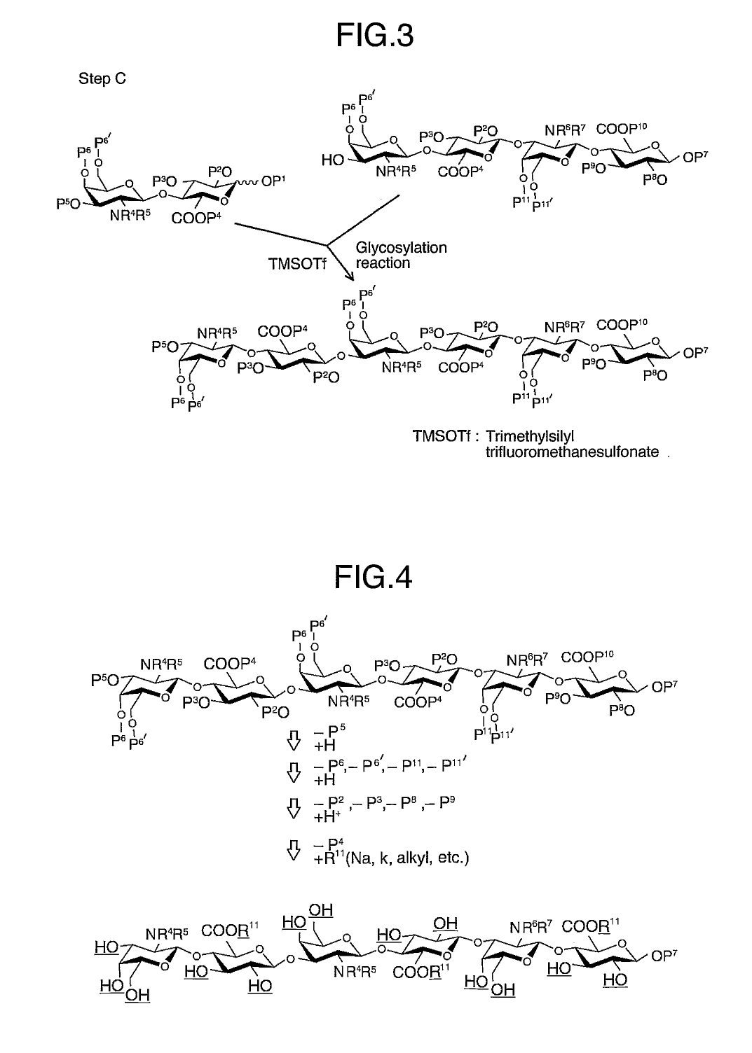

When extending further an oligosaccharide in the preparation process of the present invention, the oligosaccharide obtained in the process of the above (A) is subjected to a step of eliminating the protecting group, for example P5 in FIG. 1, at a position to be glycosylated, as shown in FIG. 2, in advance of the extension reaction which will be described in section (C).

The elimination step may be performed by selecting a suitable elimination reaction depending on the protecting group (P5) at a position to be glycosylated and other protecting groups (P2 to P4 and P6 to P11′).

For example, when levulinoyl, monochloroacetyl, etc. are used as a protecting group (P5) at a position to be glycosylated, the target protecting group can be eliminated by reacting it with for example a hydrazine acetate after dissolving the oligosaccharide obtained in the step (A) in an organic solvent such as a mixed solution of ethanol/toluene.

Usually, the elimination reaction is performed at 0 to 60° C. for 0.5 to 5 hours. In addition, in order to isolate in high yield and high purity the target oligosaccharide in which the protecting group is eliminated, the solvent is usually evaporated after the reaction and the residue is purified by gel filtration etc.

(C) Extension reaction

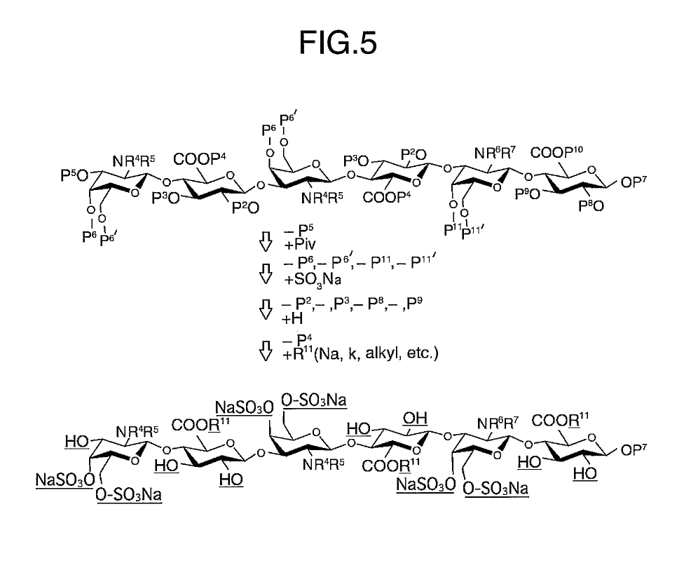

As shown in FIG. 3, according to one embodiment in the present invention, an oligoglycosaminoglycan having five or more constituent sugars can be prepared by further glycosylating the oligosaccharide from which the protecting group (P5) at a position to be glycosylated is eliminated in the above-mentioned step (B), with the same sugar donor as in step (A) in the presence of the same promoter as in step (A).

In the present invention, since the same promoter and the same sugar donor as in step (A) are used also in this step, an oligoglycosaminoglycan in which the constituent sugars have an intended chain length of five or more can be chemically synthesized in high yield.

Various conditions of this step are basically the same as those described in the step (A).

In one embodiment of the present invention, a oligoglycosaminoglycan of an intended chain length composed of 5 or more constituent sugars can be prepared by repeating the above-mentioned protecting group elimination step (B) and this extension step (C) in an intended number of times within 1 to 8. In the present invention, what is necessary is just to decide the number of times of this. reaction cycle to control the chain length of the target oligoglycosaminoglycan. Thus, the stop reaction is not necessary. In addition, only the oligoglycosaminoglycan with the desired chain length can be easily obtained. However, in order to maintain a high yield and to efficiently prepare a desired oligoglycosaminoglycan, the protecting group elimination step (B) and extension step (C) are preferably repeated in 1 to 6 times, more preferably 1 to 5 times, and particularly preferably 1 to 4 times.

(D) Elimination of all the protecting groups and selective sulfation

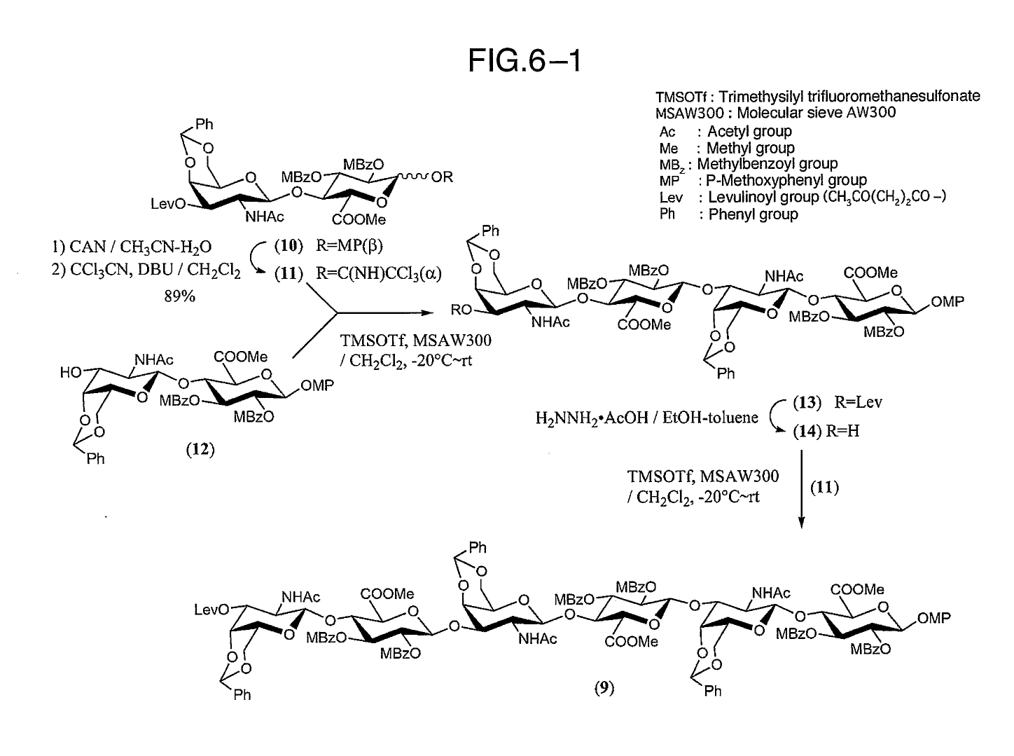

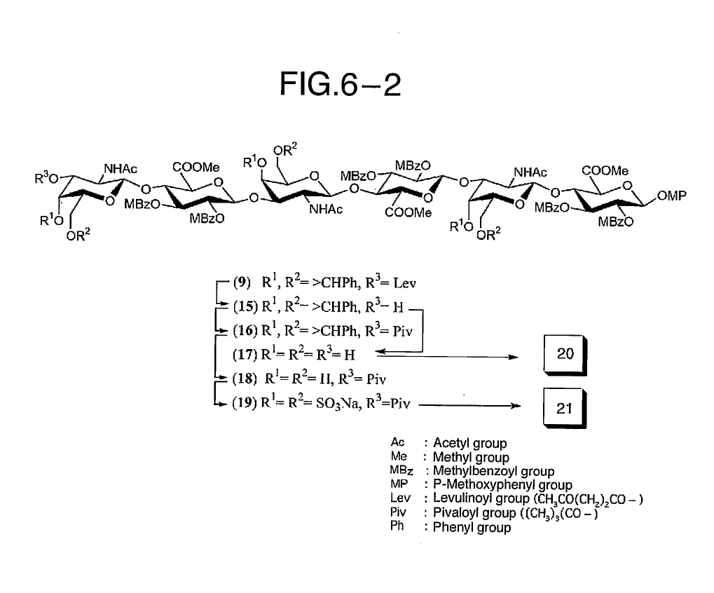

The preparation process of the present invention may further include the following steps after the above step (A) or (C): a step of eliminating all the protecting groups, for example P2 to P11′ in FIG. 1, of the oligosaccharide obtained in these steps, as shown in FIG. 4; or a steps of eliminating all the protecting groups, for example P2 to P11′ in FIG. 1, of the oligosaccharide obtained in the above step (A) or (C) and selectively sulfating at the particular position each constituent sugar: for example, FIGS. 5 and 11 represent Na sulfate binding to the hydroxyl groups at the 4th and 6th positions.

The elimination of the protecting group of the oligosaccharide obtained in the above step (A) or (C) may be performed by using a conventional method and it is desirable to eliminate the protecting group by a suitable reaction procedure depending on the kind of the above-mentioned protecting group.

For example, when the protecting group is levulinoyl, the target protecting group can be eliminated by reacting it for example with hydrazine acetate after dissolving the oligosaccharide obtained at the step (A) or (C) in an organic solvent such as a mixed solution of ethanol/toluene. When the protecting group is benzylidene, alkoxybenzylidene or cyclohexylidene, the protecting group can be eliminated by dissolving the oligosaccharide obtained at the step (A) or (C) or the oligosaccharide further subjected to an additive protecting group elimination step in a mixed solution of dichloromethane/methanol and the like and then hydrolyzing that with an acid such as camphorsulfonic acid, an acetic acid or a hydrochloric acid.

An acyl group such as acetyl and benzoyl can be removed by hydrolysis using an alkali such as lithium hydroxide and sodium hydroxide in a solvent such as aqueous tetrahydrofuran.

In order to eliminate all the protecting groups of the oligosaccharide obtained at the step (A) or (C) and to selectively sulfate the hydroxyl group at the specific position in each constituent sugar, it is desirable to perform sulfation by selectively eliminating only the protecting group at the position to be sulfated, with the other hydroxyl groups being protected.

Specifically, in order to selectively sulfate each N-acylgalactosamine at the 4th and 6th positions, the process of the above step (A) or steps (A) to (C) are preformed, for example, by using a sugar donor and sugar acceptor in which the 4th and 6th positions of each N-acylgalactosamine are protected by at least any one of benzylidene, alkoxybenzylidene and cyclohexylidene, and all the other hydroxyl groups and the carboxyl groups of each glucuronic acid derivative are protected with an alkyl group or an acyl group.

Subsequently, when the constituent sugar at the non-reducing end of the oligosaccharide obtained in the step (A) or (C) is a N-acylgalactosamine, the hydroxyl groups except for the 4th and 6th positions are substituted with a pivaloyl group.

For example, when substituting the protecting group such as levulinoyl with a pivaloyl group, an oligosaccharide having the protecting group such as levulinoyl is subjected to the reaction for example with a hydrazine acetate after dissolving the oligosaccharide in an organic solvent such as a mixed solution of ethanol/toluene to elimate the protecting group such as levulinoyl and then the resulted compound is subjected to the reaction with pivaloyl chloride in the presence of a catalyst such as N,N-dimethylaminopyridine after dissolving it for example in pyridine.

Subsequently, benzylidene, alkoxybenzylidene and/or cyclohexylidene of the pivaloylized oligosaccharide are eliminated to selectively deprotect N-acylgalactosamine at the 4th and 6th positions.

For example, the elimination of benzylidene can be carried out by dissolving the oligosaccharide to be sulfated in a mixed solution of dichloromethane/methanol and the like, and then hydrolyzing the oligosaccharide with an acid such as camphorsulfonic acid, acetic acid, and hydrochloric acid.

The sulfation may be performed for example by dissolving for example a target oligosaccharide deprotected at a desired position of the N-acylgalactosamine constituent sugar, for example, at 4th and the 6th positions as mentioned above, in a solvent such as dimethylformaldehyde and then reacting the oligosaccharide, for example, with a sulfur trioxide-trimethylamine complex. The reaction temperature during this reaction is usually 0 to 100° C., and the reaction time is usually 12 to 72 hours.

Other matters of the protecting group elimination step is similar to those as described above. In order to isolate the target oligosaccharide in high yield and high purity, it is desirable to evaporate the solvent after each step has ended and to purify the residue for example by gel filtration.

(E) intermediate

Next, the unique intermediate of oligoglycosaminoglycan obtained by the above process is described.

An embodiment of the intermediate of the present invention is represented by the general formula (4′):

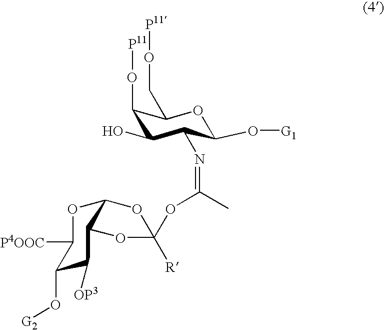

wherein R′ is selected from the group consisting of an alkyl group, an alkenyl group, an aralkyl group and an aryl group, and preferably is selected from the group consisting of phenyl, methylphenyl, ethylphenyl, propylphenyl, dimethylphenyl, methoxylphenyl, ethoxylphenyl and dimethoxylbenzoyl;

P³ is selected from the group consisting of a hydrogen atom, an alkyl group, an alkenyl group such as allyl, an aralkyl group, an aryl group and a silyl group optimally substituted with an alkyl group or an alkoxy group such as trimethylsilyl;

P⁴ is selected from the group consisting of an alkyl group, an alkenyl group such as allyl and an aralkyl group;

P¹¹ and P^{11′} are the same or independently selected from the group consisting of a hydrogen atom, an alkyl group, an alkenyl group such as allyl, an aralkyl group, an aryl group, a silyl group optinally substituted with an alkyl group or an alkoxy group such as trimethylsilyl and an alkylidene group and include one in which the two thereof are cross-linked; and

G¹ is selected from the group consisting of a hydrogen atom, an alkyl group, an aralkyl group, an alkenyl group such as allyl, an aryl group and a compound represented by the following general formula (4-1): 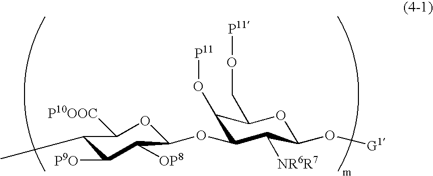
wherein m is an integer of 0 to 4;
R⁶ and R⁷ are the same or independently selected from the group consisting of a hydrogen atom, an alkyl group, an alkenyl group such as allyl, an acyl group and a phthaloyl group;
P⁸ and P⁹ are the same or independently selected from the group consisting of a hydrogen atom, an alkyl group, an alkenyl group such as allyl, an aralkyl group, an aryl group, an acyl group and a silyl group optinally substituted with an alkyl group or an alkoxy group such as trimethylsilyl;
P¹⁰ is selected from the group consisting of an alkyl group, an alkenyl group such as allyl and an aralkyl group;
P^{11 and P}^{11′} are the same or independently selected from the group consisting of a hydrogen atom, an alkyl group, an alkenyl group such as allyl, an aralkyl group, an aryl group, a silyl group optinally substituted with an alkyl group or an alkoxy group such as trimethylsilyl and an alkylidene group and include one in which the two thereof are cross-linked; and
G^{1′} is selected from the group consisting of a hydrogen atom, an alkyl group, an alkenyl group such as allyl, an aralkyl group, an aryl group and a compound represented by the following general formula (4-1′): 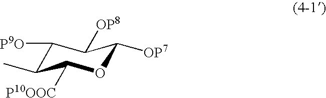
wherein P⁷ is selected from the group consisting of an alkyl group, an aralkyl group, an alkenyl group such as allyl and an aryl group;
P⁸ and P⁹ are the same or independently selected from the group consisting of a hydrogen atom, an alkyl group, an alkenyl group such as allyl, an aralkyl group, an aryl group, an acyl group and a silyl group optinally substituted with an alkyl group or an alkoxy group such as trimethylsilyl; and
P¹⁰ is selected the group consisting of an alkyl group, an alkenyl group such as allyl and an aralkyl group; and

G² is selected from the group consisting of a hydrogen atom, an alkenyl group such as allyl, an acyl group, an aralkyl group, a silyl group optinally substituted with an alkyl group or an alkoxy group such as trimethylsilyl and a compound represented by the following general formula (4-2): 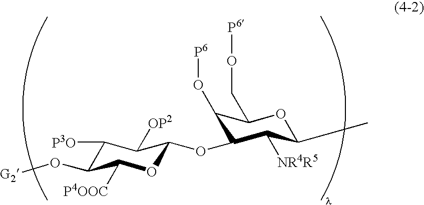
wherein λ is an integer of 0 to 4;
R⁴ and R⁵ are the same or independently selected from the group consisting of a hydrogen atom, an alkyl group, an alkenyl group such as allyl, an acyl group and a phthaloyl group;
P² and P³ are the same or independently selected from the group consisting of a hydrogen atom, an alkyl group, an alkenyl group such as allyl, an aralkyl group, an aryl group and a silyl group optinally substituted with an alkyl group or an alkoxy group such as trimethylsilyl;
P⁴ is selected from the group consisting of an alkyl group, an alkenyl group such as allyl and an aralkyl group;
P⁶ and p^{6′} are the same or independently selected from the group consisting of a hydrogen atom, an alkyl group, an alkenyl group such as allyl, an aralkyl group, an aryl group, a silyl group optinally substituted with an alkyl group or an alkoxy group such as trimethylsilyl and an alkylidene group; and
G^{2′} is selected from the group consisting of a hydrogen atom, an alkenyl group such as allyl, an acyl group, an aralkyl group, a silyl group optinally substituted with an alkyl group or an alkoxy group such as trimethylsilyl and a compound represented by the following general formula (4-2′): 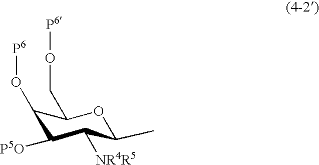
wherein R⁴ and R⁵ are the same or independently selected from the group consisting of a hydrogen atom, an alkyl group, an alkenyl group such as allyl, an acyl group and a phthaloyl group;
P⁵ is selected from the group consisting of an alkenyl group such as allyl, an acyl group, an aralkyl group and a silyl group optinally substituted with an alkyl group or an alkoxy group such as trimethylsilyl; and
P⁶ and P^{6′} are the same or independently selected from the group consisting of a hydrogen atom, an alkyl group, an alkenyl group such as allyl, an aralkyl group, an aryl group, a silyl group optinally substituted with an alkyl group or an alkoxy group such as trimethylsilyl and an alkyldene group.

The intermediates are an ortho ester type cation intermediate stabilized by a counter ion of Lewis acid and an intermediate in which the ortho ester type cation intermediates further binds to the oxygen atom in the acetamide group of anther donor or acceptor.

These intermediates enable a desired oligoglycosaminoglycan and an analogue thereof to be obtained highly stereoselectively in high yield and in high purity due to its stabilized ortho ester structure.

(F) Novel oligoglycosaminoglycan

Next, the novel oligoglycosaminoglycan of the present invention is described, which is obtained by the above chemical sythesis methods.

The novel oligoglycosaminoglycan of the present invention relates to a reducing end glucuronic acid type oligochondroitin or a sulfate thereof etc. and is represented by the following general formula (4):

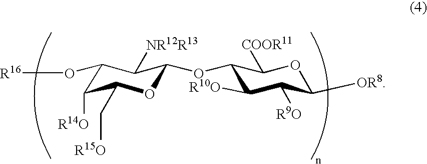

In the above general formula (4);

n is an integer of 2 to 10;

R⁸ represents a hydrogen atom or a protecting group;

R⁹ to R¹¹ are the same or independently represent a hydrogen atom or a protecting group, respectively;

R¹² and R¹³ are the same or independently selected from the group consisting of a hydrogen atom, an alkyl group, an alkenyl group such as allyl, an acyl group optionally substituted with a halogen, and a phthaloyl group, preferably selected from the group consisting of acetyl, haloacetyl, benzoyl and phthaloyl, respectively,

R¹⁴ and R¹⁵ are the same or independently represent a hydrogen atom, or a sulfate or phosphate group in which the hydrogen atom is optionally substituted with any one selected from the group consisting of sodium, potassium, copper, calcium, iron, manganese, zinc, ammonium, barium and lithium; and

R¹⁶ represents a hydrogen atom, a protecting group, or a glucronic acid or iduronic acid derivative represented by the following general formula (5):

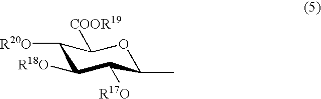

wherein, R¹⁷, R¹⁸ and R¹⁹ are the same as R⁹ to R¹¹ of the above-mentioned general formula (4) and R²⁰ is the same as R⁹ of the above-mentioned general formula (4).

In the reducing end glucuronic acid type oligochondroitin or the sulfate thereof etc. of the present invention, there is no limitation on the protecting group represented by R⁸, R⁹ to R¹¹ and R¹⁶ in the above-mentioned formula (4), and the protecting group may include, for example, a methoxyphenyl group.

Moreover, in term of the shedding inducing ability to a CD44 as described below, preferably at least one of R¹⁴ and R¹⁵ of the above-mentioned general formula (4) is a sulfate group, and particularly preferably both of R¹⁴ and R¹⁵ are sulfate groups.

As a salt of the reducing end glucuronic acid type oligochondroitin represented by the general formula (4) or the reducing end glucuronic acid type oligochondroitin sulfate or a derivative thereof, a metal salt thereof is preferable and particularly potassium and sodium salts thereof are preferable.

As described above, the reducing end glucuronic acid type oligochondroitin or the sulfate thereof etc. of the present invention always has a glucuronic acid or derivative thereof at a reducing end and thus has a specific structure which cannot be attained by the enzymatic cleaving process. Moreover, the reducing end glucuronic acid type oligochondroitin or the sulfate thereof etc. of the present invention is free from contamination of a lipid, a protein, etc. which are other components in a living body. It is further different from those obtained by the enzymatic cleaving process in that it can have only one particular chain length.

Furthermore, as the result by the preparation process of the present invention, the oligochondroitin or sulfate thereof composed of five or more constituent sugars, which cannot be obtained by the conventional chemical synthesis process, can be provided for the first time. The novel oligochondroitin or the sulfate thereof would thus greatly contribute to researches in this field.

Next, the medical application of the reducing end glucuronic acid type oligochondroitin or the sulfate thereof etc. of the present invention is described.

The reducing end glucuronic acid type oligochondroitin or the sulfate thereof etc. of the present invention has a high physiology activity in the shedding inducing ability to a CD44 molecule.

Therefore, it can be used as an active ingredient for improving, treating or preventing a diseases or condition induced by a CD44 molecule.

Specifically, the reducing end glucuronic acid type oligochondroitin or the sulfate thereof etc. of the present invention per se can be used as a medicament for improving, treating or preventing a diseases or condition induced by participation of a CD44 molecule. Of course, according to the present invention, a pharmaceutical composition can also be provided which contains the reducing end glucuronic acid type oligochondroitin or the sulfate thereof etc. of the present invention together with the pharmacologically acceptable carrier.

The medicament of the present invention is widely applicable to a diseases or condition induced by participation of a CD44 molecule and specifically it can be used for improving, treating, or preventing, for example, an autoimmune disease such as chronic articular rheumatism, systemic lupus erythematosus, multiple sclerosis, Shogren syndrome, Hashimoto disease, Addison's disease and type 1 diabetes; for example, an arthritis such as osteoarthrosis, psoriatic arthritis, lumbago, periarthritis humeroscapularis, temporomandibular arthrosis or peritendinitis; for example, an allergic diseases such as allergic rhinitis, pollinosis, syncope, hives, atopic dermatitis or bronchial asthma, or a cancer; or for immunity regulation, or for inducing cell differentiation or cell apotosis.

As shown by the Example below, particularly the above oligocondroitin sulfate sulfated at the 4th and 6th positions of N-acylgalactosamine has a large shedding inducing ability to a CD44 molecule, and thus the medical treatment effect thereof over the above-mentioned applications is large.

No particular limitations are imposed on the pharmaceutical composition of the present invention about an administration and a dosage form. Thus, it may be prepared depending on an administration form such as oral, transdermal, absorption, and injection which includes intramuscular administration, intradermal administration, hypodermical administration, intravenous administration, intracavitary administration, administration into an eye and intraperitoneal injection. A dosage form may include an injection agent, a capsule agent, a granule agent, a powder medicine, a tablet, a liquid medicine, a liposome agent, an ointment agent, a gel agent, a spray agent, an inhalation powder medicine, an applying-eyewash agent, and an eye ointment agent. Usually, the dose for an adult is 0.1 to 1000 mg per day, but it may be suitably varied depending on the weight, condition, and the like of the patient. The pharmaceutical composition of the present invention may contain other ordinary ingredients such as an excipient, a binder, a lubricant agent, a colorant, a sweetener and a disintegrating agent. Moreover, other autoimmune disease treatment agent, arthritis treatment agent, allergic disease treatment agent, immunity regulation agent, cell differentiation inducing agent, or cell apoptosis inducing agent can also be contained as an active ingredient.

Hereinafter, Example of the present invention is described in order to explain the present invention in detail. However, the present invention should not be limited at all by the following Examples.

### EXAMPLES

In the Examples shown below, a treatment applied in each step and analysis of the compound obtained in each step is performed on conditions below.

(1) Angle of rotation

Measured at 22±3° C. by HORIBA SEPA-200.

(2) ¹H NMR

Measured at 500 MHz by JEOL ECP. Me₄Si was used as an internal standard: t-BuOH=1.23 ppm to heavy water. As for the chemical shift, for example, the proton combined with C-1 of the sugar residue 3 was expressed as 1³.

(3) Silica gel chromatography

As silica gels, used were Silica Gel C-200 and C-300 from Wako Pure Chemical Co. and Silica Gel 60N (neutral, globular shape, 40 to 100 μm) from Kanto Chemistry Co. As the gel filtration carriers, used were Sephadex LH-20 and LH-60 from Amersham Biosciences.

(4) Molecular Sieve (MS)

As molecular sieve, the molecular sieve from GL Science was used after dried under reduced pressure at 180° C.

(5) Thin-layer chromatography

Silica Gel F₂₅₄ from Merck Co. is used and a toluen/ethyl acetate or ethyl acetate/methanol solvent is used.

### Example 1

### Synthesis of Reducing End Gluclone Acid Type Olygoccondoloitin Sulfate Composed of 6 Constitute Sugar

Preparation process of β-D-GalNAc-(1->[4]-β-D-GlcA-(1->3)-β-D-GalNAc-(1->)₂4)-β-D-GlcA-(1->OMP (20) or β-D-GalNAc(4,6-di-OSO₃Na)-(1->[4]-β-D-GlcA-(1->3)-β-D-GalNAc(4,6-di-OSO₃Na)-(1->₂4)-β-D-GlcA-(1->OMP (21)

The reaction process of this Example is specifically described below referring to FIGS. 6-1 and 6-2 showing the outline thereof.

(A) Synthesis of reducing end glucuronic acid type oligochondroitin composed of four constituent sugars

(A-1) Preparation of sugar donor

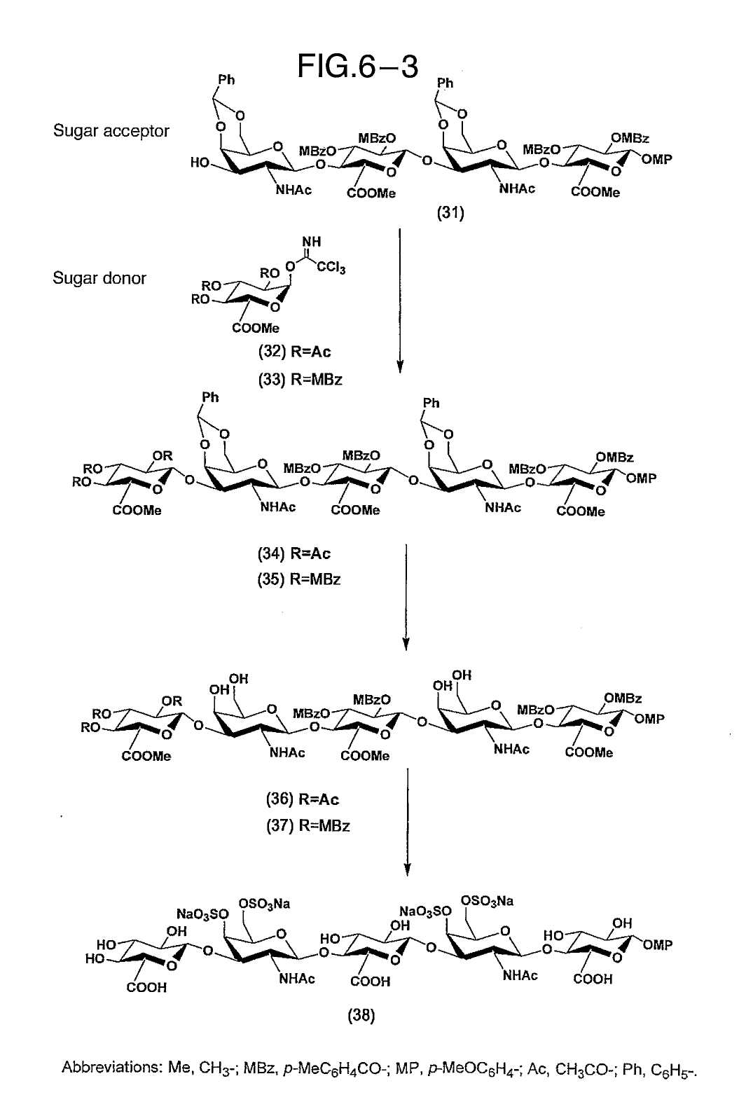

The compound composed of two constituent sugars represented by formula (10) in FIG. 6-1 has been well known and was prepared according to the process described in J. Tamura et al., Carbohydr. Res., 305, and 43-63(1998). Briefly, the compound was obtained by glycosylating a sugar donor composed of a single sugar and a sugar acceptor composed of single sugar and then imidating the protecting groups of the resulted compound.

The compound (172.7 mg, 0.184 mmol) composed of two constituent sugars represented by formula (10) was dissolved in a mixed solution of acetonitrile (CH₃CN, 8 mL) and water (2 mL), diammonium cerium (IV) nitrate (CAN, 500 mg) was added thereto, and the solution was agitated for 1 hour.

After the reaction ended, the reaction solution was diluted with chloroform (CHCl₃) and a saturated saline solution. The organic layer was washed with a saturated saline solution, was dried over an anhydrous magnesium sulfate, and, after filtration thereof, the solvent was evaporated under reduced pressure. The residual substance was purified by a silica gel column chromatography (silica gel 60N, a globular shape, neutral, 10 g, toluene/ethyl acetate 3:2 to 1:5, or ethyl acetate/methanol 50:1), and a hemiacetal compound (125.7 mg) was thus obtained.

Subsequently, the obtained hemiacetal compound is diluted with dichloromethane (CH₂Cl₂, 5 mL). Trichloroacetonitrile (CCl₃CN and 0.5 mL) was added thereto and 1,8-diazabicyclo[5.4.0]undeca-7-ene (one drop) was added thereto at 0° C. under agitation. After 30 minutes, CCl₃CN (0.2 mL) was added thereto at room temperature and the agitation was continued for 10 more minutes. The reaction solution was purified by a silica gel column chromatography (C-200, 30 g, toluene/ethyl acetate 2:1 to 1:50), and the disaccharide compound (131.1 mg) of formula (11) was obtained in yield of 73% (Rf value: 0.58 (ethyl acetate/methanol 10:1)). This compound was used for the glucosylation reaction as a sugar donor without any more purification.

(A-2) Preparation of Sugar acceptor

The compound composed of two constituent sugars represented by formula (12) in FIG. 6-1 has been well known and was prepared according to the process described in J. Tamura et al., Carbohydr. Res., 305, 43-63 (1998). Briefly, the compound was obtained by glycosylating a sugar donor composed of a single sugar and a sugar acceptor composed of single sugar and then performing deprotection.

(A-3) Synthesis of tetrasaccharide: compound of formula (13)

Dried molecular sieves AW300 (MSAW300 (5 g)) were added to a CH₂Cl₂ (43 mL) solution of the sugar donor (1.18 g, 1.21 mmol) represented by the formula (11) and the sugar acceptor (858.6 mg, 1.02 mmol) represented by the formula (12), as mentioned above, and the solution was agitated at room temperature for 1 hour. This solution was cooled to −20° C. and trimethylsilyl trifluoromethylsulfonate (TMSOTf, 213 μL, 1.18 mmol) was added thereto under agitation. The temperature of the reaction solution was gradually raised to room temperature, triethylamine and a saturated sodium bicarbonate solution were added to the reaction solution one day after, and this solution was diluted with CHCl₃.

The insolubles were filtered, the organic layer was washed with a saturated saline solution, and, after dried over anhydrous magnesium sulfate and filtrated, the solvent was evaporated under reduced pressure. The residual substance was purified by a gel filtration (LH-20, CHCl₃/methanol 1:1) and a silica gel column chromatography (C-200, 30 g, toluene/ethyl acetate 2:1-1:2), and the compound (1.20 g, 71%) of the formula (13) was thus obtained. Physical-properties data agreed with the data shown in J. Tamura et al., Carbohydr. Res., 305, and 43-63 (1998)].

(B) Removal of the levulinoyl group of tetrasaccharide, the compound of formula (13)

Hydrazine acetate (40.0 mg, 434 μmol) was added to an ethanol/toluene 5:1 solution (5 mL) of the compound (71.8 mg, 43.3 μmol) composed of four constituent sugars represented by the formula (13) obtained as mentioned above, and the solution was agitated for 20 minutes at room temperature. The solvent was evaporated under reduced pressure and the residual substance was purified by a gel filtration (LH-20, CHCl₃/methanol 1:1) to obtain the compound of the formula (14) having the following physical properties in yield of 92% (62.0 mg).

Physical Properties:

(1) Rf: 0.32 (ethyl acetate/methanol 40:1)

(2) [α]_{D}+13° (c 0.43, CHCl₃)

(3) Elemental analysis

a) Calculated value: (C₈₃H₈₆N₂O₂₈.H₂O) C, 63.18; H, 5.63; N, 1.78

b) Observed value: C, 62.90; H, 5.52; N, 1.71

c) ¹H NMR data (CDCl₃): δ 7.89-7.81 (8H, m, Ar), 7.48-7.47 (2H, m, Ar), 7.36-7.27 (8H, m, Ar), 7.19-7.13 (6H, m, Ar), 7.10-7.06 (2H, m, Ar), 6.89-6.87 (2H, m, Ar), 6.75-6.73 (2H, m, Ar), 5.73 (1H, bt, J=8.87 Hz, H-31), 5.61 (1H, dd, J_{2,3}=5.27, J_{3,4}=8.02 Hz, H-33), 5.54 (1H, d, J_{2,NH}=7.79 Hz, NH4), 5.49 (1H, dd, J_{1,2}=6.88, J_{2,3}=8.94 Hz, H-21), 5.49 (1H, s, PhCH), 5.44 (1H, d, J_{2,NH}=6.18 Hz, NH2), 5.31 (1H, s, PhCH), 5.22 (1H, t, J_{1,2}=5.27 Hz, H-23), 5.18 (1H, d, H-11), 5.17 (1H, d, J_{1,2}=8.24 Hz, H-14), 5.02 (1H, d, H-13), 4.68 (1H, dd, J_{2,3}=11.00, J_{3,4}=3.67 Hz; H-34), 4.64 (1H, brt, J=10.08 Hz, H-43), 4.53 (1H, brt, J=8.83 Hz, H-41), 4.29 (1H, s, H-44), 4.28 (1H, d, J_{1,2}=10.31 Hz, H-12), 4.25 (1H, d, J_{4,5}=10.31 Hz, H-53), 4.22 (1H, d, J_{4,5}=9.16 Hz, H-51), 3.85 (1H, m, H-6a4), 3.84 (1H, brs, H-42), 3.78 (1H, m, H-6a2), 3.75 (1H, m, H-22), 3.74, 3.72, 3.72 (3Hx3, 3s, 2 COOMe, MeOPh), 3.69 (1H, m, H-6b4), 3.52 (1H, m, H-6b2), 3.34 (1H, m, H-32), 3.25 (1H, m, H-24), 3.03 (1H, s, H-54), 2.68 (1H, s, H-52), 2.37, 2.35, 2.34, 2.30 (3Hx4, 4s, 4 MePh), 1.91 (3H, s, MeCO), 1.71 (3H, s, MeCO).

(C) Synthesis of hexasaccharide, the compound of formula (a) by the extension reaction

MSAW300 (700 mg) was added to a CH₂Cl₂ (7 mL) solution of the compound (144.3 mg and 0.148 mmol) represented by the formula (11) and the compound (160.6 mg and 0.103 mmol) represented by the formula (14) in FIG. 6-1, and the solution was agitated at room temperature for 1 hour. This solution was cooled to −20° C. and TMSOTf (19 μL, 0.11 mmol) was added thereto under agitation. The temperature of the reaction solution was gradually raised to room temperature, triethylamine and a saturated sodium bicarbonate solution were added to the reaction solution one day after, and the solution was diluted with CHCl₃.

The insolubles were filtered, the organic layer was washed with a saturated saline solution, and, after dried over anhydrous magnesium sulfate and filtered, the solvent was evaporated under reduced pressure. The residual substance was purified by a gel filtration (LH-60, CHCl₃/methanol 1:1) and a silica gel column chromatography (C-300, 6 g, toluene/ethyl acetate 1:1-1:1.5, ethyl acetate/methanol 100:1) to obtain the compound (161.4 mg, 66%) of the formula (9) having the following physical properties.

Physical Properties:

(1) Rf: 0.42 (ethyl acetate / methanol 40:1)

(2) [α]_{D}+24° (c 0.56, CHCl₃)

(3) Elemental analysis

a) Calculated value: (C₁₂₆H₁₃₁N₃O₄₃.2H₂O) C, 62.75; H, 5.65; N, 1.74

b) Observed value: C, 62.89; H, 5.52; N, 1.72%

c) ¹H NMR data (CDCl₃): δ 7.96 (2H, d, J=8.02 Hz, Ar), 7.91-7.88 (3H, m, Ar), 7.85-7.80 (6H, m, Ar), 7.62 (1H, d, J=7.33 Hz, Ar), 7.48 (1H, d, J=6.41 Hz, Ar), 7.38-7.35 (2H, m, Ar), 7.32-7.21 (17H, m, Ar), 7.18-7.13 (5H, m, Ar), 7.06 (2H, d, J=8.02 Hz, Ar), 6.90-6.86 (2H, m, Ar), 6.76-6.73 (2H, m, Ar), 5.73 (1H, t, J_{2,3}=J_{3,4}=8.93 Hz, H-31), 5.67 (1H, s, PhCH), 5.60-5.56 (2H, m, H-33, 35), 5.49 (1H, s, PhCH), 5.48 (1H, dd, J_{1,2}=7.33 Hz, H-21), 5.46 (1H, d, J_{2,NH}=7.56 Hz, NH6), 5.26 (1H, s, PhCH), 5.17 (1H, m, H-23), 5.17 (2H, d, H-11, 16), 5.04-4.98 (3H, m, H-13, 15, 25), 4.83 (1H, dd, J_{3,4}=7.56, J_{4,5}=10.54 Hz, H-45), 4.65 (2H, m, H-43, 36), 4.62 (1H, d, J_{2,NH}=8.71 Hz, NH4), 4.51 (1H, brt, J=9.05 Hz, H-41), 4.46 (1H, d, J_{3,4}=3.44 Hz, H-44), 4.34 (1H, d, J_{3,4}<5.5 Hz, H-46), 4.32 (1H, d, J_{4,5}=10.77 Hz, H-55), 4.30 (1H, dd, J_{2,3}=10.31, J_{3,4}=3.36 Hz, H-32), 4.23 (1H, d, J_{4,5}=9.85 Hz, H-53), 4.20 (1H, d, J_{4,5}=9.17 Hz, H-51), 4.14 (1H, d, J_{1,2}=8.71 Hz, H-12), 4.04 (1H, bt, J=9.91 Hz, H-22), 3.91 (2H, d, J=12.15 Hz, H-6a2, 6a4), 3.88 (1H, d, H-42), 3.87 (1H, m, H-34), 3.86 (1H, m, NH2), 3.83 (1H, d, J_{gem}=6.65 Hz, H-6a6), 3.75-3.66 (2H, m, H-14, 24), 3.72 (1H, m, H-6b4), 3.72, 3.71, 3.65 (3Hx4, 3s, 3 COOMe, MeOPh), 3.69 (1H, m, H-6b6), 3.53 (1H, d, J_{gem}=8.25 Hz, H-6b2), 3.24 (1H, m, H-26), 2.97 (1H, s, H-56), 2.78(1H, s, H-54),2.70-2.41 (4H, m, 2CH₂), 2.52 (1H, s, H-52), 2.43, 2.40, 2.38, 2.37, 2.34, 2.30 (18H, 6s, 6 MePh), 2.03 (3H, s, COCH₃),1.80, 1.77, 1.72 (3Hx3, 3s, 3NAc).

(D) Removal of the levulinoyl group of hexasaccharide, the compound of formula (9)

As shown in FIG. 6-2, hydrazine acetate (12.6 mg, 28.2 μmol) was added to an ethanol/toluene 4:1 solution (2.5 mL) of the compound (30.8 mg, 13.0 μmol) represented by the formula (9), and the solution was agitated for 1 hour at room temperature. The solvent was evaporated under reduced pressure and the residual substance was purified by a gel filtration (LH-20, CHCl₃/methanol 1:1) to obtain the compound of the formula (15) (29.9 mg) having the following physical properties.

Physical Properties:

(1) Rf: 0.43 (ethyl acetate methanol 10:1)

(2) [α]_{D}+19° (c 0.68, CHCl₃)

(3) Elemental analysis

a) Calculated value: (C₁₂₁H₁₂₅N₃₀₄₁Na, [M+Na]+)2298.77

b) Observed value: 2298.94

c) ¹H NMR data (CDCl₃): δ 7.91-7.79 (12H, m, Ar), 7.54 (2H, d, J=7.11 Hz, Ar), 7.45-7.44 (2H, m, Ar), 7.37-7.28 (12H, m, Ar), 7.23-7.12 (9H, m, Ar), 7.06 (2H, d, J=8.25 Hz, Ar), 6.89-6.87 (2H, m, Ar), 6.75-6.74 (2H, m, Ar), 5.72 (1H, bt, J=8.82 Hz, H-31), 5.60 (1H, dd, J_{2,3}=3.90, J_{3,4}=7.55 Hz, H-35), 5.58 (1H, dd, J_{2,3}=6.18, J_{3,4}8.24 Hz, 33), 5.56 (1H, s, PhCH), 5.48 (1H, dd, J_{1,2}=7.10, J_{2,3}=8.94 Hz, H-21), 5.44 (1H, s, PhCH), 5.40 (1H, d, J_{2,NH}=6.63 Hz, NH6), 5.33 (1H, s, PhCH), 5.20 (1H, bt, J=5.83 Hz, H-23), 5.17 (1H, d, J_{1,2}=8.25 Hz, H-16), 5.16 (1H, d, H-11), 5.08 (1H, bt, J=4.01 Hz, H-25), 5.00 (1H, d, J_{1,2}=4.12 Hz, H-15), 4.96 (1H, d, J_{1,2}=5.73 Hz, H-13), 4.92 (1H, d, J_{2,NH}=6.88 Hz, NH2), 4.75 (1H, d, J_{2,NH}=8.70 Hz, NH4), 4.73 (1H, dd, J_{4,5}=10.54 Hz, H-45), 4.69 (1H, d, J_{1,2}=8.48 Hz, H-14), 4.63 (1H, dd, J_{2,3}=11.00, J hd 3,4=3.44 Hz, H-36), 4.60 (1H, dd, J_{4,5}=8.02 Hz, H-43), 4.50 (1H, brt, J=8.82 Hz, H-41), 4.31 (1H, d, J_{3,4}=4.12 Hz, H-44), 4.29 (1H, d, H-55), 4.22 (1H, s, H-46), 4.19 (2H, d, J=9.39 Hz, H-51, 53), 4.13 (1H, d, J_{1,2}=8.48 Hz, H-12), 4.04 (1H, m, H-34), 3.89 (1H, d, J_{gem}=12.37 Hz, H-6a4), 3.86 (1H, d, J_{gem}=12.61 Hz, H-6a2), 3.84 (1H, d, J_{gem}=11.45 Hz, H-6a6), 3.84 (1H, d, J_{3,4}=3.66 Hz, H-42), 3.75 (1H, m, H-22), 3.73, 3.70, 3.65 (3Hx4, 3s, 3 COOMe, MeOPh), 3.65 (2H, m, H-6b4, 6b6), 3.54 (1H, d, H-6b2), 3.54 (1H, m, H-24), 3.23 (1H, ddd, H-26), 3.15 (1H, m, H-32), 2.96 (1H, s, H-56), 2.82(1H, s, H-54), 2.59 (1H, s, H-52), 2.40, 2.38, 2.37, 2.35, 2.34, 2.30 (3Hx6, 6s, 6 MePh), 1.85, 1.70, 1.66 (3Hx3, 3s, 3NAc).

(E) Removal of other protecting groups, benzylidene and acyl

Camphorsulfonic acid (4.2 mg) was added to a CH₂Cl₂/methanol 1:1 solution (1.6 mL) of the above compound (17.8 mg, 7.82 μmol) represented by the formula (15), and the solution was agitated at room temperature for 12 hours. Camphorsulfonic acid (6.6 mg) was added thereto, and the solution was agitated at room temperature for further 24 hours. Excessive amount of diisopropylethylamine was added to the reaction solution, the solvent was evaporated under reduced pressure and the residual substance was purified by a gel filtration (LH-20, CHCl₃/methanol 1:1) to obtain the compound of the formula (17) in yield of 78% (12.2 mg) of.

After it was confirmed by ¹H NMR that benzylidene had been removed from the compound of the formula (15), the compound (5.4 mg) of a formula (17) was dissolved in a tetrahydrofuran/water 15:1 solution (1.6 mL) and 1.25M lithium hydroxide solution (45 μL) was added thereto at 0° C. under agitation. The solvent was evaporated under reduced pressure for one hour, methanol (1.5 mL) was added to the residual substance, and 0.1M sodium methoxide (0.5 mL) was dropped to this solution under agitation. The reaction was stopped with acetic acid of 50% three days after, and the solvent was evaporated under reduced pressure. A residual substance was purified by a gel-filtration (LH-20, 1% acetic acid), and the compound represented by the following general formula (20) was obtained in yield of 92% of (3.3 mg):

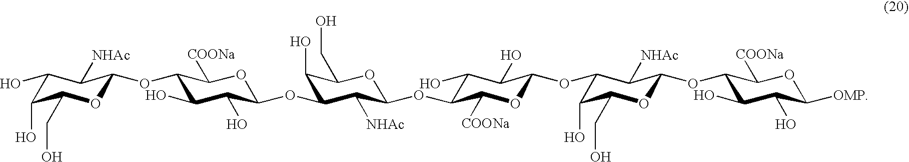

The physical properties of the obtained compound are shown below.

Physical Properties:

(1) Rf: 0.32 (n-butanol/acetic acid/water 1:1:1)

(2) [α]_{D}+20° (c 0.33, water)

(3) Elemental analysis

a) Calculated value: (C₄₉H₅₈N₃₀₃₅Na, [M+Na−3H]2−) 640.68

b) Observed value: 640.69

c) ¹H NMR data (D₂O): δ 7.09 (2H, d, J=9.17 Hz, Ph), 6.96 (2H, d, J=8.94 Hz, Ph), 5.07 (1H, d, J_{1,2}=7.79 Hz, H-11), 4.54 (1H, d, J_{1,2}=7.79 Hz, H-13 or 15), 4.53 (2H, d, J_{1,2}=7.56 Hz, H-12 or 14, 15 or 13), 4.49 (1H, d, J_{1,2}=8.48 Hz, H-14 or 12), 4.45 (1H, d, J_{1,2}=8.24 Hz, H-16), 4.09-4.07 (3H, m, H-42, 44, 46), 4.05-4.00 (1H, bt, J=9.63 Hz, H-22 or 24), 3.98 (1H, bt, J=9.74 Hz, H-24 or 22), 3.91-3.66 (19H, m, H-31, 41, 51, 32, 52, 62x2, 43, 53, 34, 54, 64x2, 45, 55, 36, 56, 66x2), 3.85 (1H, m, H-26), 3.80 (3H, s, MeOPh), 3.62 (1H, bt, J=9.32 Hz, H-33 or 35), 3.61 (2H, bt, J=8.83 Hz, H-21, 35 or 33), 3.36, 3.34 (2H, m, H-23, 25), 2.02 (3H, s, MeCO), 1.99 (6H, s, MeCO).

(F) Selective sulfation: synthesis of the compound of formula (21)

As shown in FIG. 6-2, pivaloyl chloride (30 μL) and catalytic amount of N,N-dimethylaminopyridine were added to a pyridine solution (1.5 mL) of the compound (28.7 mg, 12.6 μmol) represented by the formula (15), and the solution was agitated at 80° C. for 3 hours. Pivaloyl chloride (90 μL) was added thereto and the solution was agitated at 80° C. for 2 hours. The reaction solution was returned to room temperature, excessive amount of methanol was added thereto, the reaction solution was purified by a gel filtration (LH-20, CHCl₃/methanol 1:1) to obtain the compound (27.0 mg, 89%) of the formula (16).

Subsequently, camphorsulfonic acid (2.9 mg) was added to a CH₂Cl₂/methanol 1:1 solution (1 mL) of the compound (8.6 mg, 3.6 μmol) represented by the formula (16), and the solution was agitated at room temperature for 20 hours. Excessive amount of diisopropylethylamine was added to the reaction solution, the solvent was evaporated under reduced pressure and the residual substance was purified by a gel filtration (LH-20, CHCl₃/methanol 1:1) to obtain the compound (8.1 mg) of the formula (18).

Subsequently, sulfur trioxide-trimethylamine complex (60 mg) was added to a dimethylformamide solution (0.5 mL) of the compound (8.1 mg, 3.6 μmol) represented by the formula (18), and the solution was agitated at 57° C. for 22 hours. The sulfur trioxidetrimethylamine complex (59 mg) was added thereto, and the solution was agitated at 57° C. for 26 hours. The reaction solution was cooled to room temperature, the reaction solution was purified with a gel filtration (LH-20, CHCl₃/methanol 1:1) and an ion-exchange resin [Dowex 50W (Na⁺), methanol/water 8:1] to obtain the compound (9.6 mg, 91%) of the formula (19).

Finally, the compound (9.6 mg) of the formula (19) was dissolved in a mixed solution of tetrahydrofuran (0.5 mL) and water (0.04 mL), and 1.25M lithium hydroxide solution (0.2 mL) was added thereto at 0° C. under agitation. The agitation was continued at room temperature overnight. The solvent was evaporated under reduced pressure and methanol (0.5 mL), CH₂Cl₂ (0.15 mL) were added to the residual substance, and 0.5M sodium hydroxide (0.3 mL) was dropped to this solution under agitation. The reaction was stopped with acetic acid of 50% after 22 hours, and the solvent was evaporated under reduced pressure. The residual substance was purified by a gel filtration (LH-20, 1% acetic acid), and the compound represented by the following general formula (21) was obtained in yield of 54% (3.7 mg):

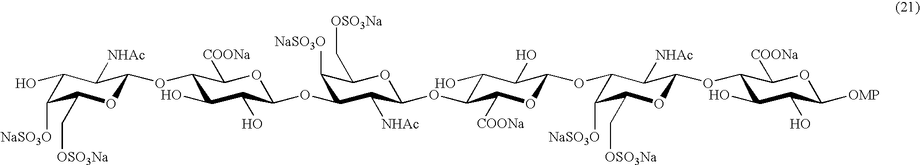

The physical properties of the obtained compound are shown below.

Physical Properties:

(1) Rf: 0.14 (n-butanol:acetic acid:water 1:1:1)

(2) [α]_{D}−8.1 (c 0.37, water)

(3) Elemental analysis

a) Calculated values (C₄₉H₆₂N₃₀₃₅Na₆, [M−3Na]3−) 623.33,

b) Observed values 623.34

c) ¹H NMR data (D₂O): δ 7.09 (2H, d, J=9.17 Hz, Ar), 6.96 (2H, d, J=9.17 Hz, Ar), 5.12 (1H, d, J_{1,2}=7.79 Hz, H-11), 4.79 (2H, s, H-42, 44), 4.71 (1H, s, H-46), 4.65 (2H, m, H-12, 14), 4.60 (1H, d, J_{1,2}=8.02 Hz, H-16), 4.56, 4.55 (2H, 2d, J_{1,2}=7.79 Hz, H-13, 15), 4.32-4.21 (6H, m, H-62x2, 64x2, 66x2), 4.18 (1H, d, J_{4,5}=9.85 Hz, H-51), 4.11 (3H, m, H-52, 54, 56), 4.05-4.01 (3H, m, H-22, 24, 34 or 32), 4.00, 3.99 (2H, 2d, J_{4,5}=9.62 Hz, H-53, 55), 3.93-3.78 (7H, m, H-31, 41, 32 or 34, 43, 45, 26, 36), 3.80 (3H, s, MeOPh), 3.66 (1H, bt, J=8.47 Hz, H-33 or 35), 3.66 (1H, bt, J=9.05 Hz, H-35 or 33), 3.63(1H, bt, J=9.17 Hz, H-21), 3.41(1H, bt, J=10.77 Hz, H-23 or 25), 3.40 (1H, bt, J=10.20 Hz, H-25 or 23), 2.02 (3H, s, MeCO), 2.00 (6H, s, 2MeCO).

### Comparative Example 1

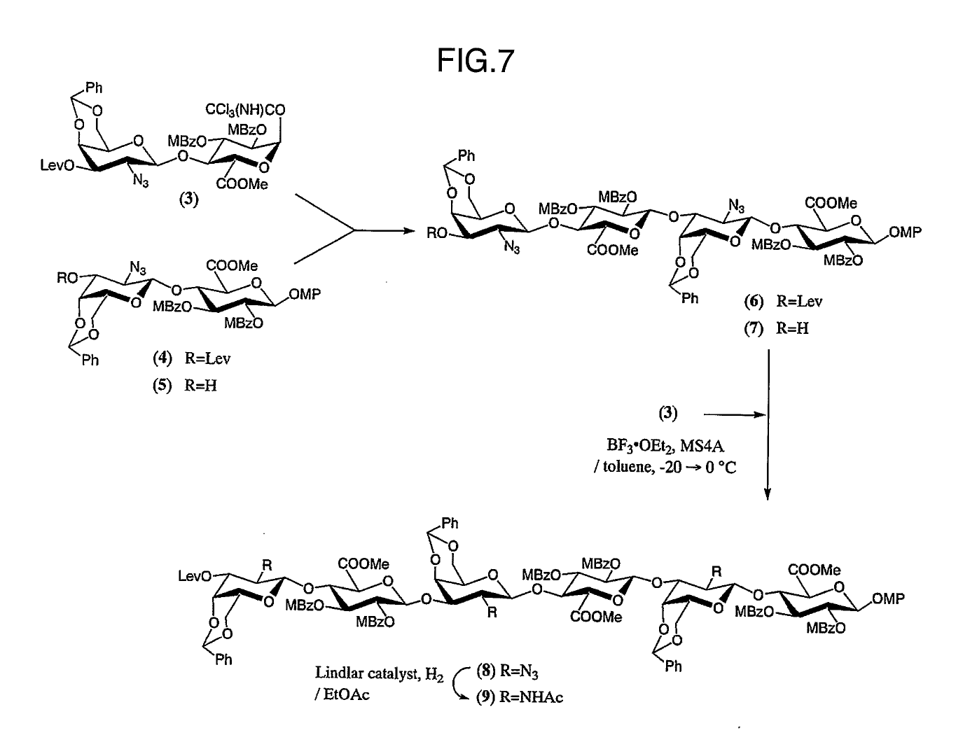

As shown in FIG. 7, a reducing end glucuronic acid type oligochondroitin composed of six constituent sugars and a sulfate thereof were prepared in the same manner as in Example 1 except that the glycosylation reaction was carried out using an azidized sugar donor and sugar acceptor represented by formulas (3) and (4) in FIG. 7 in place of the sugar donor and sugar acceptor of the above-mentioned Example 1 in the presence of BF₃O-Et₂ in place of the promoter of the above-mentioned Example 1, and that the reaction product represented by the formula (8), which is obtained by the extension reaction of the compound represented by the formula (6) with the sugar donor, were hydrogenized and reduced in the presence of a Lindlar catalyst in ethyl acetate to perform N-acetylation.

### Referential Example

Another preparation process using thioacetic acid in place of the Lindlar catalyst used in the preparation process of the above-mentioned Comparative Example 1 has been proposed in Bio. Med. Chem. Lett. 1995; 5(13): 1351-1354.

### RESULTS

According to the preparation process of Example 1, in which trimethylsilyl trifluoromethylsulfonate (TMSOTf) was used as a promoter, and, after reducing the azides in the stage of disaccharide, glycosylation of each sugar chain unit was performed; the compound composed of four constituent sugars represented by the formula (13) in FIG. 6-1 was obtained in a high yield of 71% and a reducing end glucuronic acid type oligochondroitin and reducing end glucuronic acid type oligochondroitin sulfate composed of six constituent sugars represented by the formula (20) and (21) in FIG. 6-2 were obtained in a high yield of 66%.

On the other hand, in the preparation process of Comparative Example 1 in which BF₃-OEt₂ was used as a promoter, an azide type sugar donor and an azide type sugar acceptor were glycosylated to perform an extension reaction and, in the stage of tetrasaccharide or hexasaccharide, the azide groups were reduced into N-acetyl group; when subjecting the azidized tetrasaccharide to a reduction reaction, the yield was 50%, which was lower than Example 1 by 20% or more. When subjecting the azidized hexasaccharide (6) to the reduction reaction, conversion to N-acety group did not occur and the target compound of the formula (9) was not obtained.

In the referential process, when subjecting the azidized tetrasaccharide to the reduction reaction, the yield was 43%, which was lower than the process of Example 1 by about 30%.

### Example 2

### Synthesis of Reducing End Glucuronic Acid Type Oligochondroitin Composed of 5 Constituent Sugars

(A) Preparation of pentasaccharide:

Preparation of Methyl (2,3,4-tri-O-acetyl-β-D-glucopyranosyluronate)-(1→3)-(2-acetamido-4,6-O -benzylidene-2-deoxy-β-D-galactopyranosyl)-(1→4)-(methyl 2,3-di-O-(4-methylbenzoyl)-β-D-glucopyranosyluronate)-(1→3)-(2-acetamido-4,6-O-benzylidene-2-deoxy-β-D-galactopyranosyl)-(1→4)-(4-methoxyphenyl 2,3-di-O-(4-methylbenzoyl)-β-D-glucopyranosid)uronate (34)

(A-1 and A-2) Sugar donor and sugar acceptor The sugar acceptor (31) shown in FIG. 6-3 was prepared according to J. Tamura & M. Tokuyoshi, Biosci. Biotech. Biochem., 68, 2436-2443 (2004) and similarly the sugar donor (32) was prepared according to J.-C. Jacquinet, Carbohydr. Res., 199, 153-181 (1990).

(A-3) Synthesis of pentasaccharide, the compound of formula (34)

As shown in FIG. 6-3, MSAW300 (464 mg) was added to a dichloromethane solution (8.3 mL) of a sugar acceptor (31) (207.4 mg, 0.133 mmol) and a sugar donor (23) (205.6 mg, 0.430 mmol) and the resulted solution was agitated at room temperature for 1 hour. The reaction solution was cooled to −20° C., TMSOTf (23 μl, 0.13 mmol, 0.3 equivalent to the sugar donor (32)) was added thereto, and the solution was agitated overnight continuously raising the temperature to room temperature. After the reaction ended, a celite filtration was performed on the solution, the filtrate was extracted with CHCl₃. The organic layer was washed with a saturated sodium bicarbonate solution and a saturated saline solution, dried over anhydrous magnesium sulfate and filtered. Then, the filtrate was condensed, and was purified by a gel filtration (LH-20, CHCl₃:MeOH=1:1) to obtain a crude product. This product was further purified with a silica gel column (Hexane:EtOAc=2:1-1:20-MeOH:EtOAc=1:80-1:60) to obtain a syrup-like compound of the formula (34) having the following physical properties (69.6 mg, 37.1 μmol) in a yield of 28%.

Physical Properties:

(1) [α]_{D}=+21.1° (c 1.05, CHCl₃).

(2) Elemental analysis

a) Calculated valuses: C₉₆H₁₀₂N₂O₃₇.H₂O; C, 60.91; H, 5.55; N, 1.48%. b) Observed value: C, 60.81; H, 5.53; N, 1.48%.

c) ¹H-NMR (CDCl₃): δ7.85-7.80 (m, 8H, Ph), 7.47-7.28 (m, 10H, Ph), 7.18-7.05 (m, 8H, Ph), 6.87 (m, 2H, Ph), 6.74 (m, 2H, Ph), 5.72 (brt, 1H, J=8.83 Hz, H-3¹), 5.59 (brt, 1H, J_{2,3}=6.41, J_{3,4}=8.24 Hz, H-3³), 5.47 (dd, 1H, J_{1,2}=6.87, J_{2,3}=8.94 Hz, H-2¹), 5.40 (s, 1H, PhCH), 5.39 (d, 1H, J=7.10 Hz, NH⁴), 5.32 (s, 1H, PhCH), 5.32 (br, 1H, NH²), 5.24 (t, 1H, J_{1,2}=J_{2,3}=6.41 Hz, H-2³), 5.20-5.13 (m, 4H, H-1¹, 14, 45, 35), 4.96 (d, 1H, J_{1,2}=6.42 Hz, H-1³), 4.91 (brt, 1H, J=8.02 Hz, H-25), 4.84 (d, 1H, J_{1,2}=8.25 Hz, H-1²), 4.77 (d, 1H, J_{1,2}=7.33 Hz, H-1⁵), 4.63 (dd, 1H, H-3⁴), 4.56 (brt, 1H, H-4³), 4.50 (t, 1H, J_{3,4}=J_{4,5}=8.94 Hz, H-4¹), 4.23 (br, 1H, H-32), 4.23 (d, 1H, J_{3,4}=3.20 Hz, H-4⁴), 4.19 (d, 1H, H-5¹), 4.18 (d, 1H, J_{4,5}=9.62 Hz, H-5³), 4.06 (d, 1H, J_{3,4}=3.21 Hz, H-4²), 3.96 (d, 1H, J_{4,5}=9.62 Hz, H-55), 3.86 (d, 1H, J_{gem}=11.46 Hz, H-6a²), 3.85 (d, 1H, J_{gem}=12.14 Hz, H-6a⁴), 3.73, 3.71, 3.70, 3.67 (4s, 3Hx4, 4OMe), 3.64 (d, 1H, H-6b⁴), 3.60 (d, 1H, H-6b²), 3.44 (m, 1H, H-2²), 3.22 (m, 1H, H-2⁴), 2.97 (s, 1H, H-54), 2.83 (s, 1H, H-5²), 2.36, 2.34, 2.34, 2.32 (4s, 3Hx4, 4PhMe), 2.00, 1.99, 1.98 (3s, 3Hx3, 3OAc), 1.90, 1.63 (2s, 3Hx2, 2NAc).

When the reaction was performed in a similar ways except by using TMSOTf in 0.7 equivalent to the sugar donor (32), the yield of the compound of the formula (34) was 30%.

(B) Removal of protecting group:

Preparation of Methyl (2,3,4-tri-O-acetyl-β-D-glucopyranosyluronate)-(1→3)-(2-acetamido-2-deoxy-β-D-galactopyranosyl)-(1→4)-{methyl 2,3-di-O-(4-methylbenzoyl)-β-D-glucopyranosyluronate}-(1→3)-(2-acetamido-2-deoxy-β-D-galactopyranosyl)-(1→4)-{4-methoxyphenyl 2,3-di-O-(4-methylbenzoyl)-β-D-glucopyranosid}uronate (36)

The compound (34) (23.9 mg, 12.7 mmol) was dissolved in dichloromethane (1.4 mL) and methanol (1.4 mL), and camphorsulfonic acid (9.8 mg) was added thereto and the solution was agitated at room temperature overnight. After the reaction ended, triethylamine was added to neutralize the solution, and the solution is condensed by azeotrope with toluene. The concentrated residual substance was purified by a gel filtration (LH-20, CHCl₃:MeOH=1:1) to obtain a compound of the formula (36) having the following physical properties (18.2 mg, 10.7 μmo) in a yield of 84%. Removal of benzylidene was confirmed by ¹H-NMR, and the solution was used for the next step without further purification.

¹H-NMR (CD₃OD, selected): δ7.79-7.65 (m, 8H, Ph), 7.14-7.07 (m, 8H, Ph), 6.76 (m, 2H, Ph), 6.67 (m, 2H, Ph), 3.69, 3.66, 3.60, 3.57 (4s, 3Hx4, 4OMe), 2.27, 2.26, 2.24, 2.23 (4s, 3Hx4, 4PhMe), 1.92, 1.88, 1.86, 1.85, 1.20 (5s, 3Hx5, 3OAc, 2NAc).

(C) Sulfation:

Preparation of β-D-Glucopyranosyluronic acid-(1→3)-2-acetamido-2-deoxy-4,6-di-O-sulfonate-β-D-galactopyranosyl-(1→4)-β-D-glucopyranosyluronic acid-(1→3)-2-acetamido-2-deoxy-4,6-di-O-sulfonate-β-D-galactopyranosyl-(1→4)-4-methoxyphenyl β-D-glucopyranosyluronic acid, tetrasodium salt (38)

The compound (36) (18.2 mg, 10.7 μmol) was dissolved in DMF (1.2 mL), SO₃.Me₃N (119.2 mg) were added to the resulted solution, and the solution was agitated at 60° C. overnight. On the next day, SO₃.Me₃N (119.9 mg) was added to the solution keeping the temperature at 60° C., and the solution was agitated further overnight. After the reaction ended, the temperature of the solution was returned to room temperature, the solution was purified by a gel filtration (LH-20, CHCl₃:MeOH=1:1), and the sulfate compound(21.2 mg and 10.1 μmol) was eluted with Dowex AG50 (Na⁺) column in a yield of 94%. This sulfate compound was dissolved in THF (1.4 mL) and water (6 drops), and, under ice-cooled condition, 1.25N LIOH (0.6 mL) was added to the solution, and the solution was agitated overnight continuously raising the temperature to room temperature. After the reaction ended, the solution was condensed, and the concentrated residual substance was dissolved in methanol (1.4 mL) and dichloromethane (0.42 mL). 0.5N NaOH (0.8 mL) was added to the solution, and the solution was agitated at room temperature for 4 and half hours. After the reaction ended, 50% AcOH was added to neutralize the solution, and purification was performed with a gel filtration (LH-20, 1% AcOH) to obtain a compound of the formula (38) (9.2 mg, 6.28 μmo) having the following physical properties in a yield of 59% through the two steps. 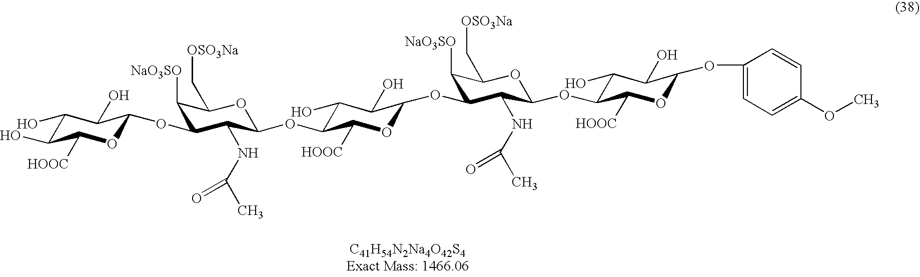
Physical properties of the compound of the formula (38) are shown below.
Physical Properties:

(1) [α]_{D}=+1.20 (c 0.52, H₂O)

(2) ¹H-NMR (D₂O): 67.10 (m, 2H, Ph), 7.09 (m, 2H, Ph), 5.12 (d, 1H, J_{1,2}=8.02 Hz, H-1¹), 4.84, 4.81 (2s, 1Hx2, H-4², 4⁴), 4.66 (m, 2H, H-1², 1⁴), 4.56 (d, 1H, J_{1,2}=7.79 Hz, H-1³), 4.53 (d, 1H, J_{1,2}=7.79 Hz, H-1⁵), 4.29-4.20 (m, 4H, H-6², 6⁴), 4.18 (d, 1H, J_{4,5}=9.85 Hz, H-5¹), 4.11 (m, 2H, H-5², 5⁴), 4.04 (m, 4H, H-2², 2⁴, 3², 3⁴), 4.00 (d, 1H, J_{4,5}=9.85 Hz, H-5³), 3.95 (d, 1H, J_{4,5}=9.85 Hz, H-5⁵), 3.91 (brt, 1H, J=9.28 Hz, H-4¹), 3.85 (brt, 1H, J=9.17 Hz, H-4³), 3.80 (m, 1H, H-3¹), 3.80 (s, 3H, OMe), 3.67 (brt, 1H, J=9.28 Hz, H-3³), 3.63 (dd, 1H, J_{2,3}=9.39 Hz, H-2¹), 3.61 (brt, 1H, J=9.61 Hz, H-4⁵), 3.48 (brt, 1H, J=9.28 Hz, H-3⁵), 3.41 (dd, 1H, J_{2,3}=9.17 Hz, H-2³), 3.37 (dd, 1H, J_{2,3}=9.16 Hz, H-2⁵), 2.01, 2.00 (2s, 3Hx2, 2NAc).

MS (ESI): m/z (M−3H+Na)²⁻ calcd. for C₄₁H₅₁N₂Na₅O₄₂S₄, 743.02; found 743.01, (M−2H)²⁻ calcd. for C₄₁H₅₂N₂Na₄O₄₂S₄, 732.03; found, 732.02; (M−2Na)²⁻ calcd. for C₄₁H₅₃N₂Na₃O₄₂S₄, 721.03; found, 721.03; (M−3H)³⁻ calcd. for C₄₁H₅₁N₂Na₄O₄₂S₄, 487.68; found, 487.67; (M−2H−Na) ³⁻ calcd. for C₄₁H₅₂N₂Na₃O₄₂S₄, 480.35; found 480.34, (M−H−, 2Na)³⁻ calcd. for C₄₁H₅₃N₂Na₂O₄₂S₄, 473.03; found 473.02.

### Example 3

### Synthesis of Reducing End Glucuronic Acid Type Oligochondroitin Composed of 5 Constituent Sugars (2)

(A) Proparation of pentasaccharide:

Preparation of Methyl {2,3,4-tri-O-(4-methylbenzoyl)-β-d-glucopyranosyluronate}-(1→3)-(2-acetamido-4,6-O-benzylidene-2-deoxy-β-D-galactopyranosyl)-(1→4)-{methyl 2,3-di-O -(4-methylbenzoyl)-β-D-glucopyranosyluronate}-(1→3)-(2-acetamido-4,6-O-benzylidene-2-deoxy-β-D-galactopyranosyl)-(1→4)-{4-methoxyphenyl 2,3-di-O-(4-methylbenzoyl)-β-D-glucopyranosid}uronate (35)

(A-1 and A-2) Sugar donor and sugar acceptor

The sugar acceptor (31) shown in FIG. 6-3 was prepared according to J. Tamura & M. Tokuyoshi, Biosci. Biotech. Biochem., 68, 2436-2443 (2004). Similarly, the sugar donor (33) was prepared according to F. Goto & T. Ogawa, Tetrahedron Lett., 33, 6841-6844 (1992).

(A-3) Synthesis of pentasaccharide (the compound of formula (35))

As shown in FIG. 6-3, MSAW300 (231.6 mg) was added to a dichloromethane solution (4.5 mL) of the sugar acceptor (31) (110.0 mg and 70.5. mol) and the sugar donor (33) (101.7 mg and 144 μmol) and the resulted solution was agitated at room temperature for 1 hour.

After the reaction solution was cooled to −20° C., TMSOTf (23 μl, 0.13 mmol, 0.7 equivalent to the sugar donor (33)) was added to the solution, and the solution was agitated overnight raising the temperature to room temperature continuously. After the reaction ended, a celite filtration was performed, the filtrate was extracted with CHCl₃. The organic layer was washed with a saturated sodium bicarbonate solution and a saturated saline solution, and dried over anhydrous magnesium sulfate and filtered. Then, the filtrate was condensed, and was purified by a gel filtration (LH-20, CHCl₃:MeOH=1:1) to obtain a crude product. This product was further purified with a silica gel column (Hexane:EtOAc=7:1-1:5-MeOH:EtOAc=1:60-1:20) to obtain a syrup-like compound of the formula (35) having the following physical properties (74.4 mg, 35.4 μmol) in a yield of 50%.

Physical properties:

(1) [α]_{D}=+23.1° c 1.34, CHCl₃).

(2) Elemental analysis

a) Calculated values: C₁₁₄H₁₁₄N₂O₃₇.1.5H₂O; C, 64.72; H, 5.59; N, 1.32%.

b) Observed value: C, 64.45; H, 5.72; N, 1.37%.

c) ¹H-NMR (CDCl₃): δ7.84-7.76 (m, 12H, Ph), 7.69 (d, 2H, J=8.25 Hz, Ph), 7.34-7.27 (m, 10H, Ph), 7.18-7.13 (m, 8H, Ph), 7.09-7.04 (m, 6H, Ph), 6.87 (m, 2H, Ph), 6.74 (m, 2H, Ph), 5.79 (brt, 1H, J=9.28 Hz, H-3⁵), 5.71 (t, 1H, J_{2,3}=J_{3,4}=8.70 Hz, H-3³), 5.59 (brt, 1H, J=9.63 Hz, H-4⁵), 5.58 (brt, 1H, J=7.79 Hz, H-3¹), 5.47 (dd, 1H, J_{1,2}=7.90 Hz, H-2³), 5.42 (dd, 1H, J_{1,2}=7.33, J_{2,3}=9.16 Hz, H-2⁵), 5.38 (brd, 1H, J=7.10 Hz, NH²), 5.36 (d, 1H, J=6.88 Hz, NH⁴), 5.31 (s, 1H, PhCH), 5.28 (s, 1H, PhCH), 5.23 (brt, 1H, J=7.12 Hz, H-2¹), 5.17 (d, 1H, J_{1,2}=8.25 Hz, H-1⁴), 5.16 (d, 1H, H-1³), 5.09 (d, 1H, H-1⁵), 4.96 (d, 1H, J_{1,2}=8.02 Hz, H-1²), 4.90 (d, 1H, J_{1,2}=6.86 Hz, H-1¹), 4.60 (dd, 1H, J_{2,3}=11.00, J_{3,4}=3.44 Hz, H-3⁴), 4.49 (t, 1H, J_{3,4}=J_{4,5}=8.70 Hz, H-4³), 4.45 (brt, 1H, J=8.60 Hz, H-4¹), 4.45 (m, 1H, H-3²), 4.24 (d, 1H, J_{4,5}=9.85 Hz, H-5⁵), 4.18 (d, 1H, H-5³), 4.13 (brs, 1H, H-4⁴), 4.13 (brs, 1H, H-4²), 4.10 (d, 1H, J_{4,5}=9.40 Hz, H-5¹), 3.84 (brd, 1H, J_{gem}=12.37 Hz, H-6a⁴), 3.81 (d, 1H, J_{gem}=12.00 Hz, H-6a²), 3.72, 3.69, 3.67, 3.56 (4s, 3Hx4, 4OMe), 3.61 (brd, 1H, J=10.77 Hz, H-6b⁴), 3.56 (m, 1H, H-6b²), 3.32 (m, 1H, H-2²), 3.18 (m, 1H, H-2⁴), 2.95 (s, 1H, H-5⁴), 2.84 (s, 1H, H-5²), 2.36, 2.34, 2.34, 2.30, 2.30, 2.28 (6s, 3Hx7, 7PhMe), 1.60, 1.56. (2s, 3Hx2, 2NAc).

When TMSOTf was used in 0.3 equivalent to the sugar donor (33), the yield of the compound of the formula (35) was 23%.

Discussions:

(1) According to the glycosylation reaction using an imidate of this invention, an imidoyloxy group is eliminated to produce a cation at the 1st position of a sugar donor. The resulted cationic intermediate and a free hydroxyl group of a sugar acceptor bind to each other, i.e. result in a glycosylation reaction. The high yield resulting from the process of Example 1 would be based on the following reasons: trimethylsilyl trifluoromethanesulfonate (TMSOTf) used as a promoter in Example 1 forms an ion pair with the cationic intermediate to stabilize the unstable cationic intermediate, and, as a result, suppresses the degradation thereof. Therefore, it is expectable that the compound included in the above-mentioned general formula (1) would have the same effect.

Further, it is considered that use of the acetamide type disaccharide unit as a sugar donor greatly contributes to the high yield.

(2) In the reaction of (11)+(14)->(9) in the above Example 1, the reaction products were subjected to a thin layer chromatography. At the time of starting the reaction, observed as main spots are the spots at Rf=0.07, 0.38, 0.49, 0.62 and 0.78, respectively and at the time of ending the reaction, those spots substantially converged to one spot at Rf=0.41.

Usually, although a glucosylation reaction may result in multi-spot by the degradation of a sugar donor, in such a case, those spots do not converge. Therefore, this results show that the reaction in Examples of the present invention results in the target condensate via two-or more intermediate.

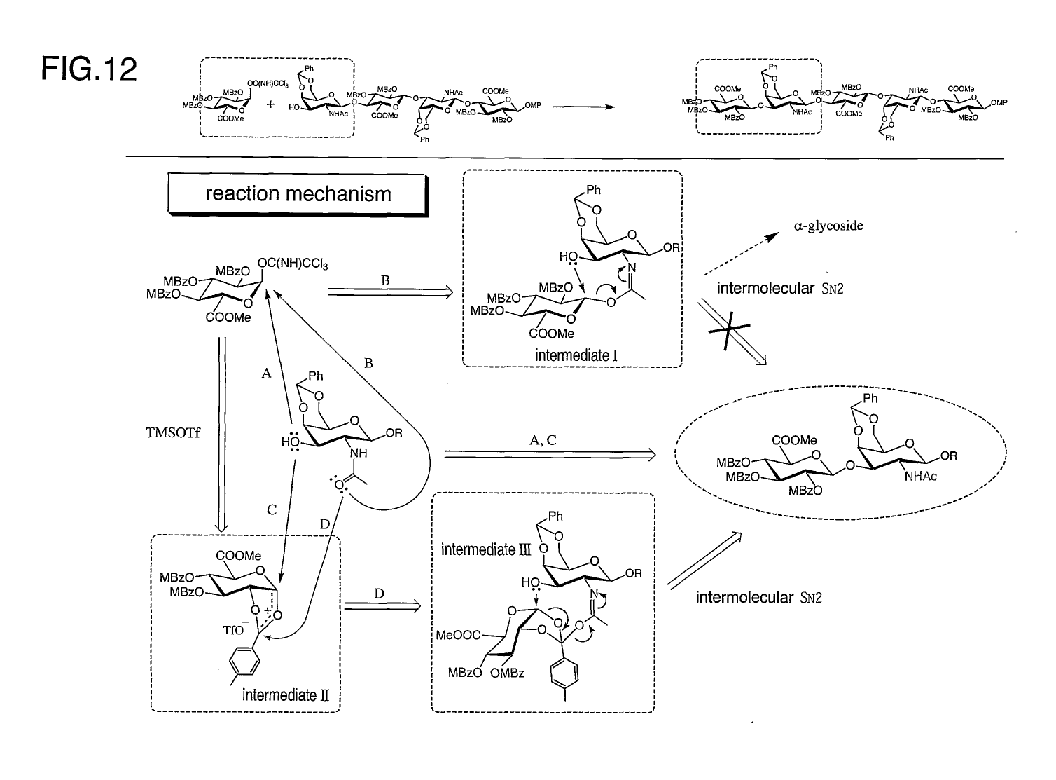

In this regard, when a glucosylation reaction is caused in the presence of a Lewis acid as shown in FIG. 12, the following paths is expected: usually the imidoyl group of the sugar donor is activated and the sugar acceptor attacks the carbon atom at the 1st position of the sugar donor which has been converted to a potential cation and the both undergo condensation (path A); or alternatively, when the hydroxyl group of the 2nd position of the sugar donor is protected with an acyl group, the carbonyl oxygen of the acyl group attacks the carbon atom at the 1st position of the activated sugar donor to form a reaction intermediate (II), the oxygen atom of the sugar acceptor attacks the carbon atom at the 1st position of the intermediate (II) and the both undergo condensation (path C).

However, both of these paths could not explain the above-mentioned multi-spot phenomenon as well as the subsequent convergence phenomenon and β-selectivity in Examples of the present invention, and the existence of a reaction path way which is different from the above A and C was thus suggested in the reaction of (11)+(14)->(9).

The present inventor considers about this phenomenon that the reaction in Examples of the present invention proceeded via the path D shown in FIG. 12. That is, in the process of the present invention, it is expected that the oxygen atom of the acetamide group of the sugar receptor attacks the carbon atom of the ortho ester of the intermediate II to form intermediate III and the carbon atom at the 1st position of this intermediate III is attacked by the oxygen atom of the sugar acceptor to synthesize the target β-selective condensate.

This expected reaction mechanism agrees with the above-mentioned multi-spot phenomenon and the phenomenon in which the multi-spots converge. That is, the multi-spot phenomenon is considered to be attributable to the existence of three types of acetamide in the sugar donor and the sugar acceptor and the existence of a number of sub groups of the intermediates III. Further, the subsequent convergence can be explained by the generation of β-selective condensate by condensation of the intermediate III and the sugar donor.

In addition, when it goes via the above-mentioned reaction path (D), the carbon atom at the 1st position of the sugar donor receives steric control and the reaction intermediate II receives an attack only from the side where ortho ester does not exist. The fact that the final product obtained by the present invention goes through this path is consistent with the result that only β-glucoside was obtained in Examples 1 and 3.

Furthermore, it has been proved from Examples 2 and 3 that the yield is improved sharply in the case where the 2nd position of the sugar donor is substituted with a benzoyl type substituent, as compared with the case that it is substituted with an acetyl type substituent: the compound of formula 35 is in a yield of 50%, while the compound of formula 34 is in a yield of 30%. This is consistent with the fact that the conjugate system of the ortho ester carbocation of the intermediate II is longer and more stable when the 2nd position of the sugar donor is protected with a benzoyl type substituent rather than an acetyl type substituent.

### Example 4

### CD44 Shedding Induction by Synthetic Oligosaccharide

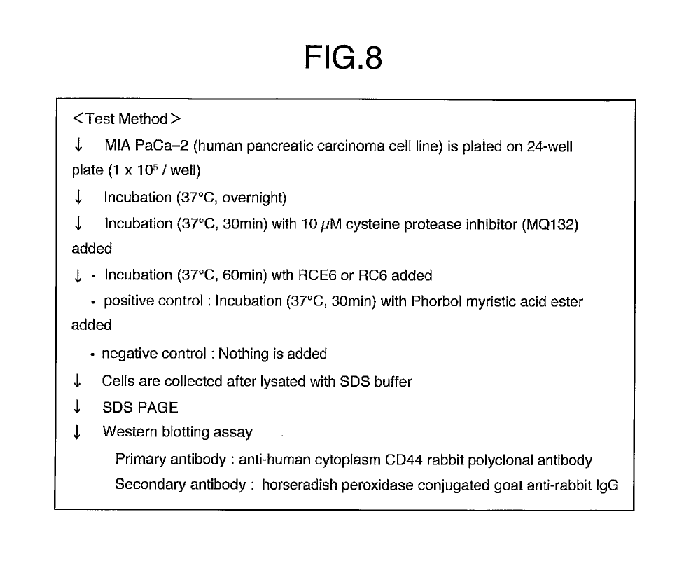

About the compound of the formula (20) and (21) obtained in Example 1, a CD44 shedding induction ability in a cancer cell was evaluated according to the test method shown in FIG. 8.

Test results

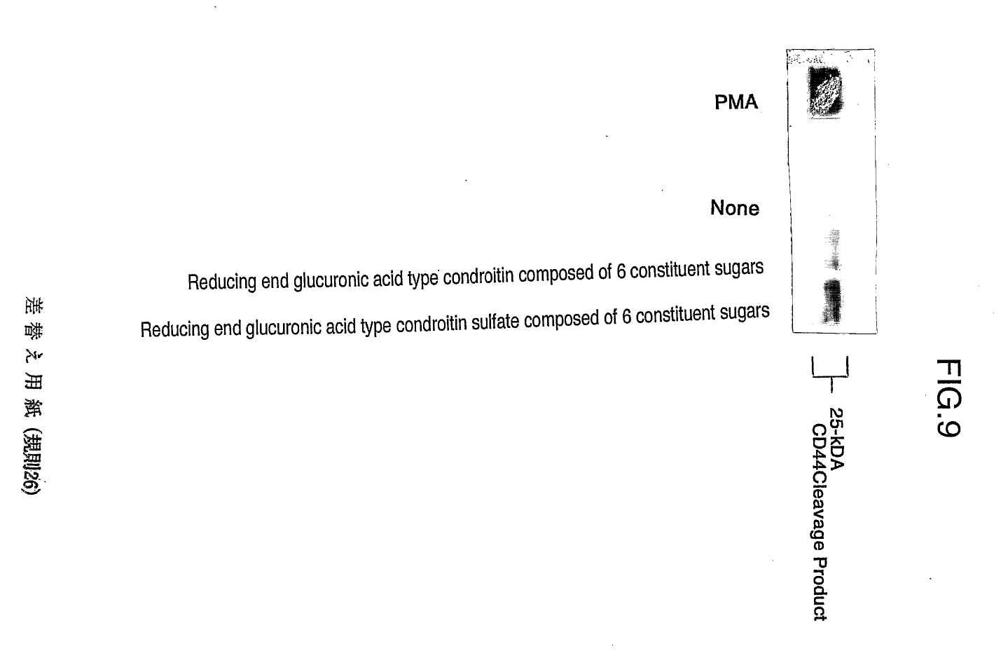

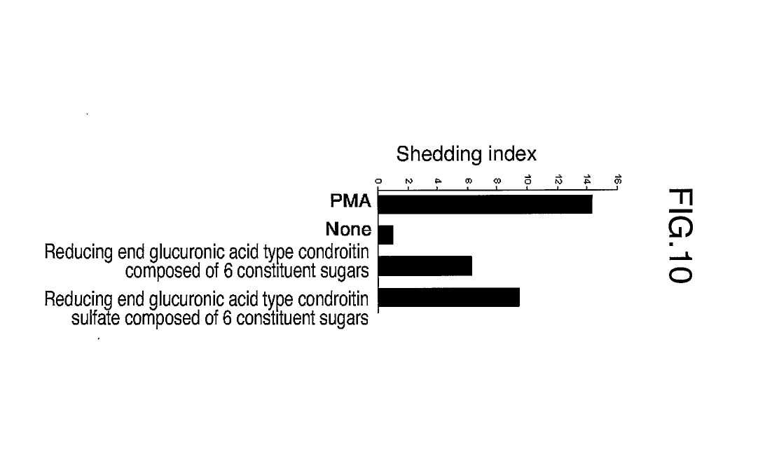

As shown in FIGS. 9 and 10, even a reducing end glucuronic acid type chondroitin composed of 6 constituent sugars not sulfated induced the shedding of a CD44. Moreover, the shedding of a CD44 was induced more strongly by a reducing end glucuronic acid type chondroitin sulfate E composed of 6 constituent sugars in which the 4th and 6th positions of N-acylgalactosamine are sulfated than a reducing end glucuronic acid type chondroitin.

This proves that the main chain itself of a reducing end glucuronic acid type chondroitin, constituting the backbone of the reducing end glucuronic acid type chondroitin sulfate E, participates in a CD44 shedding inducing ability. This proves that the sulfate group on the main chain of a reducing end glucuronic acid type chondroitin also participates in the induction of a CD44 shedding.

It is already known that a CD44 molecule participates in a variety of diseases. Therefore it is apparent that the reducing end glucuronic acid type oligochondroitin sulfate composed of six or more constituent sugars of the present invention is effective in the diseases and condition in which a CD44 molecule is involved, based on the high CD44 shedding inducing ability.

### INDUSTRIAL APPLICABILITY

As described above, according to this invention, provided be can a simple process of preparing highly stereoselectively, in high yield and in high purity an oligoglycosaminoglycan having an intended chain length and structure composed of four or more, particularly five or more constituent sugars. Moreover, according to this invention, provided be can a reducing end glucuronic acid type oligochondroitin sulfate composed of five or more intended number of constituent sugars in a high purity and a pharmaceutical composition containing the same.

1. A process for preparating an oligoglycosaminoglycan or a intermediate thereof, wherein the process comprises:
a step (A) of subjecting (A-1) a sugar donor having a glucuronic acid or iduronic acid derivative at the reducing end in which a leaving group is added to the reducing end hydroxyl group to be glycosylated and the other hydroxyl groups and the carboxyl groups are protected to the glycosylation reaction with (A-2) a sugar acceptor having a N-acylgalactosamine derivative at the non-reducing end in which the non-reducing end hydroxyl group to be glycosylated is free and the other hydroxyl groups are protected in the presence of (A-3) a Lewis acid as a promoter which is an activator for the leaving group of the sugar donor.

2. The process according to claim 1, wherein the intermediate is represented by the following general formula (4′): 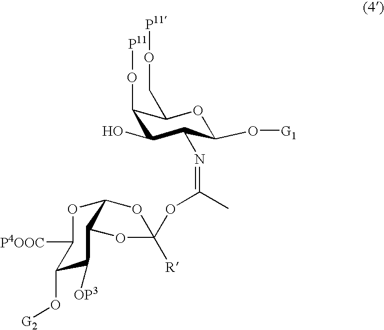
wherein R′ is selected from the group consisting of an alkyl group, an alkenyl group, an aralkyl group and an aryl group;
P³ is selected from the group consisting of a hydrogen atom, an alkyl group, an alkenyl group, an aralkyl group, an aryl group and a silyl group optinally substituted with an alkyl group or an alkoxy group;
P⁴ is selected from the group consisting of an alkyl group, an alkenyl group and an aralkyl group;
P¹¹ and P^{11′} are the same or independently selected from the group consisting of a hydrogen atom, an alkyl group, an alkenyl group, an aralkyl group, an aryl group, a silyl group optinally substituted with an alkyl group or an alkoxy group and an alkylidene group and include one in which the two thereof are cross-linked; and
G¹ is selected from the group consisting of a hydrogen atom, an alkyl group, an aralkyl group, an alkenyl group, an aryl group and a compound represented by the following general formula (4-1): 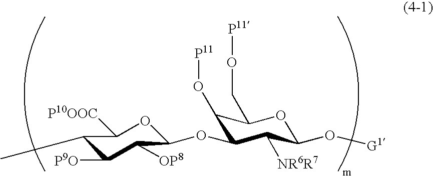
wherein m is an integer of 0 to 4;
R⁶ and R⁷ are the same or independently selected from the group consisting of a hydrogen atom, an alkyl group, an alkenyl group, an acyl group and a phthaloyl group;
P⁸ and P⁹ are the same or independently selected from the group consisting of a hydrogen atom, an alkyl group, an alkenyl group, an aralkyl group, an aryl group, an acyl group and a silyl group optinally substituted with an alkyl group or an alkoxy group;
P¹⁰ is selected from the group consisting of an alkyl group, an alkenyl group and an aralkyl group;
P¹¹ and P^{11′} are the same or independently selected from the group consisting of a hydrogen atom, an alkyl group, an alkenyl group, an aralkyl group, an aryl group, a silyl group optinally substituted with an alkyl group or an alkoxy group and an alkylidene group and include one in which the two thereof are cross-linked; and
G^{1′} is selected from the group consisting of a hydrogen atom, an alkyl group, an alkenyl group, an aralkyl group, an aryl group and a compound represented by the following general formula (4-1′): 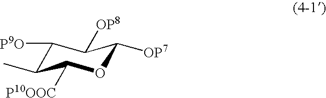
wherein P⁷ is selected from the group consisting of an alkyl group, an aralkyl group, an alkenyl group and an aryl group;
P⁸ and P⁹ are the same or independently selected from the group consisting of a hydrogen atom, an alkyl group, an alkenyl group, an aralkyl group, an aryl group, an acyl group and a silyl group optinally substituted with an alkyl group or an alkoxy group; and
P¹⁰ is selected the group consisting of an alkyl group, an alkenyl group and an aralkyl group; and
G² is selected from the group consisting of a hydrogen atom, an alkenyl group, an acyl group, an aralkyl group, a silyl group optinally substituted with an alkyl group or an alkoxy group and a compound represented by the following general formula (4-2): 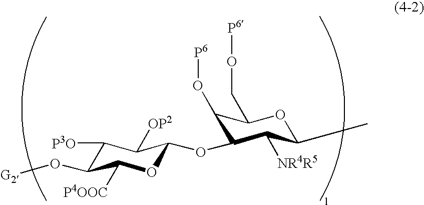
wherein λ is an integer of 0 to 4;
R⁴ and R⁵ are the same or independently selected from the group consisting of a hydrogen atom, an alkyl group, an alkenyl group, an acyl group and a phthaloyl group;
P² and P³ are the same or independently selected from the group consisting of a hydrogen atom, an alkyl group, an alkenyl group, an aralkyl group, an aryl group and a silyl group optinally substituted with an alkyl group or an alkoxy group;
P⁴ is selected from the group consisting of an alkyl group, an alkenyl group and an aralkyl group;
P⁶ and P^{6′} are the same or independently selected from the group consisting of a hydrogen atom, an alkyl group, an alkenyl group, an aralkyl group, an aryl group, a silyl group optinally substituted with an alkyl group or an alkoxy group and an alkylidene group; and
G2′ is selected from the group consisting of a hydrogen atom, an alkenyl group, an acyl group, an aralkyl group, a silyl group optinally substituted with an alkyl group or an alkoxy group and a compound represented by the following general formula (4-2′): 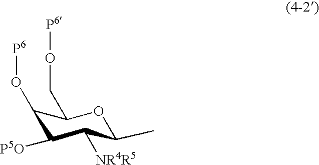
wherein R⁴ and R⁵ are the same or independently selected from the group consisting of a hydrogen atom, an alkyl group, an alkenyl group, an acyl group and a phthaloyl group;
P⁵ is selected from the group consisting of an alkenyl group, an acyl group, an aralkyl group and a silyl group optinally substituted with an alkyl group or an alkoxy group; and
P⁶ and P^{6′} are the same or independently selected from the group consisting of a hydrogen atom, an alkyl group, an alkenyl group, an aralkyl group, an aryl group, a silyl group optinally substituted with an alkyl group or an alkoxy group and an alkylidene group.

3. The process according to claim 1, wherein the promoter is a compound represented by the following general formula (1): 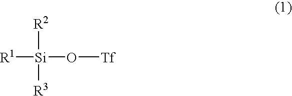
wherein R¹, R² and R³ the same or independently represent a linear or branched alkyl group or an aromatic group unsubstituted or of which at least one of the hydrogen atoms are substituted, and Tf represents a trifluoromethanesulfonyl group.

4. he process according to claim 1, wherein:
the sugar donor is a glucuronic acid or iduronic acid derivative in which a leaving group is added to the reducing end hydroxyl group to be glycosylated and the other hydroxyl groups and the carboxyl groups are protected, or an oligosaccharide derivative having as a basic constituent unit a basic disaccharide unit composed of a N-acylgalactosamine derivative and a glucuronic acid or iduronic acid derivative in which a leaving group is added to the reducing end hydroxyl group to be glycosylated and the other hydroxyl groups and the carboxyl groups are protected; and
the sugar acceptor is a glucuronic acid or iduronic acid derivative in which the non-reducing end hydroxyl group to be glycosylated is free and the other hydroxyl groups and the carboxyl groups are protected, or an oligosaccharide derivative having as a basic constituent unit a N-acylgalactosamine derivative and a glucuronic acid or iduronic acid derivative in which the reducing end hydroxyl group to be glycosylated is free and the other hydroxyl groups and the carboxyl groups are protected.

5. The process according to claim 1, further comprising the steps of:
(B) eliminating one protecting group of the non-reducing end of the oligosaccharide derivative obtained in the above step (A), and
(C) subjecting the oligosaccharide derivative from which the one protecting group is eliminated to the glycosylation reaction with the sugar acceptor in the presence of the promoter; and
after the step (A), repeating these steps in an intended number of times within 1 to 8.

6. The process according to claim 1, wherein:
the sugar donor is a chondroitin derivative represented by the following general formula (2): 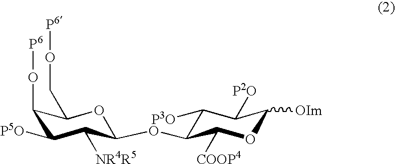
wherein R⁴ and R⁵ are the same or independently selected from the group consisting of a hydrogen atom, an alkyl group, an alkenyl group, an acyl group and a phthaloyl group;
Im is an imidoyl optionally substituted with a halogen;
P² and P³ are the same or independently selected from the group consisting of a hydrogen atom, an alkyl group, an alkenyl group, an aralkyl group, an aryl group and a silyl group optinally substituted with an alkyl group or an alkoxy group;
P⁴ is selected from the group consisting of an alkyl group, an alkenyl group and an aralkyl group;
P⁵ is selected from the group consisting of an alkenyl group, an acyl group, an aralkyl group and a silyl group optinally substituted with an alkyl group or an alkoxy group; and
P⁶ and P^{6′} are the same or independently selected from the group consisting of a hydrogen atom, an alkyl group, an alkenyl group, an aralkyl group, an aryl group, a silyl group optinally substituted with an alkyl group or an alkoxy group and an alkylidene group; and
the sugar acceptor is a reducing end glucuronic acid type chondroitin derivative represented by the following general formula (3): 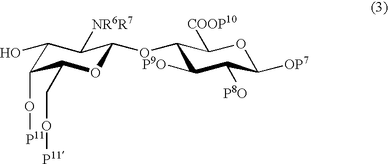
wherein R⁶ and R⁷ are the same or independently selected from the group consisting of a hydrogen atom, an alkyl group, an alkenyl group, an acyl group and phthaloyl group;
P⁷ is selected from the group consisting of an alkyl group, an aralkyl group, an alkenyl group and an aryl group;
P⁸ and P⁹ are the same or independently selected from the group consisting of a hydrogen atom, an alkyl group, an alkenyl group, an aralkyl group, an aryl group, an acyl group and a silyl group optinally substituted with an alkyl group or an alkoxy group and an acyl group;
P¹⁰ is selected from the group consisting of an alkyl group, an alkenyl group and an aralkyl group; and
P¹¹ and P^{11′} are the same or independently selected from the group consisting of a hydrogen atom, an alkyl group, an alkenyl group, an aralkyl group, an aryl group, a silyl group optinally substituted with an alkyl group or an alkoxy group and an alkylidene group and include one in which the two thereof are cross-linked.

7. The process according to claim 1, wherein:
the sugar donor is a chondroitin derivative represented by the following general formula (2′): 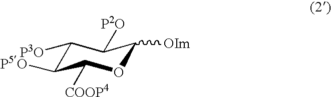
wherein Im is an imidoyl group optionally substituted with a halogen;
P² and P³ are the same or independently selected from the group consisting of a hydrogen atom, an alkyl group, an alkenyl group, an aralkyl group, an aryl group and a silyl group optinally substituted with an alkyl group or an alkoxy group;
P⁴ is selected from the group consisting of an alkyl group, an alkenyl group and an aralkyl group; and
P^{5′} is selected from the group consisting of an alkenyl group, an acyl group, an aralkyl group and a silyl group optinally substituted with an alkyl group or an alkoxy group; and
the sugar acceptor used is a reducing end glucuronic acid type chondroitin derivative represented by the following general formula (3): 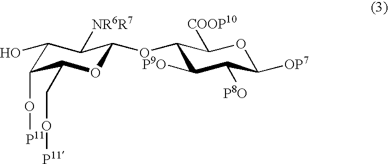
wherein R⁶ and R⁷ are the same or independently selected from the group consisting of a hydrogen atom, an alkyl group, an alkenyl group, an acyl group and a phthaloyl group;
P⁷ is selected from the group consisting of an alkyl group, an aralkyl group, an alkenyl group and an aryl group;
P⁸ and P⁹ are the same or independently selected from the group consisting of a hydrogen atom, an alkyl group, an alkenyl group, an aralkyl group, an aryl group, an acyl group and a silyl group optinally substituted with an alkyl group or an alkoxy group and an acyl group;
P¹⁰ is selected from the group consisting of an alkyl group, an alkenyl group and an aralkyl group; and
P¹¹ and P^{11′} are the same or independently selected from the group consisting of a hydrogen atom, an alkyl group, an alkenyl group, an aralkyl group, an aryl group, a silyl group optinally substituted with an alkyl group or an alkoxy group and an alkylidene group and include one in which the two thereof are cross-linked.

8. The process according to claim 1, wherein the substituent at the 2nd position of the glucuronic acid or iduronic acid derivative in the sugar donor is protected by an acyl group which stabilizes an ortho ester.

9. The process according to claim 3, wherein R¹, R² and R³ are independently a hydrogen atom, or a linear or branched alkyl group.

10. The process according to claim 1, wherein the promoter is trimethylsilyl trifluoromethanesulfonate (TMSOTf).

11. The process according to claim 1, wherein the steps (B) and (C) are repeated in one to five times.

12. The process according to claim 1, further comprising a step (D-1) in which all the protecting groups of the oligosaccharide derivative obtained in the step (A) or (C) are eliminated.

13. The process according to claim 1, further comprising a step (D-2) in which all the protecting groups of the oligosaccharide derivative obtained in the step (A) or (C) are eliminated, and each N-acylgalactosamine are selectively sulfated at the 4th and 6th positions thereof.

14. The process according to claim 1, wherein the sugar donor is a compound represented by the general formula (2) or (2′) and the sugar acceptor is the reducing end glucuronic acid type chondroitin derivative represented by the general formula (3);
wherein, if the oligosaccharide derivative obtained in the step (A) or (C) has an N-acylgalactosamine derivative at the non-reducing end, the hydroxyl groups at a position other than the 4th and 6th positions of the N-acylgalactosamine derivative are protected with a pivaloyl group;
the groups protecting the 4th and 6th positions of each N-acylgalactosamine derivative are eliminated; and
the deprotected oligosaccharide is sulfated to selectively sulfate the 4th and 6th positions of each N-acylgalactosamine.

15. The process according to claim 14, the protecting group is benzylidene, alkoxybenzylidene or cyclohexylidene.

16. An oligoglycosaminoglycan intermediate compound represented by the general formula (4′): 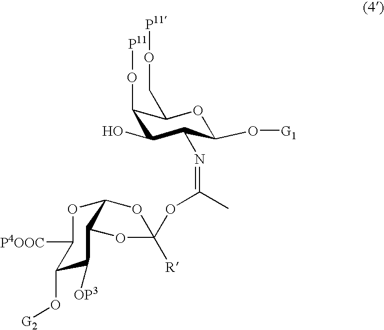
wherein R′, P³, P⁴, P¹¹, P^{11′}, G¹ and G² are the same as those defined in claim 2.

17. A reducing end glucuronic acid type oligochondroitin or a reducing end glucuronic acid type oligochondroitin sulfate, or a salt or derivative thereof represented by the following general formula (4): 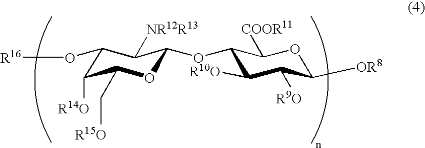
wherein n is an integer of 2 to 10;
R⁸ represents a hydrogen atom or a protecting group;
R⁹ to R¹¹ are the same or independently represent a hydrogen atom or a protecting group;
R¹² and R¹³ are the same or independently selected from the group consisting of a hydrogen atom, an alkyl group, an alkenyl group, an acyl group and a phthaloyl group;
R¹⁴ and R¹⁵ the same or independently represent a hydrogen atom, or a sulfate or phosphate group optionally substituted with any one selected from the group consisting of sodium, potassium, copper, calcium, iron, manganese, zinc, ammonium, barium and lithium; and;
R¹⁶ represents a hydrogen atom, or a glucronic acid or iduronic acid derivative represented by the following general formula (5): 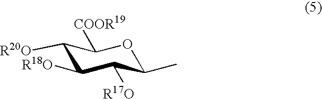
wherein R¹⁷, R¹⁸ and R¹⁹ represent the same or independently a hydrogen atom or a protecting group, or sodium, potassium, copper, calcium, iron, manganese, zinc, ammonium, barium or lithium; and
R²⁰ represents a hydrogen atom or a protecting group.

18. The reducing end glucuronic acid type oligochondroitin sulfate, or the salt or derivative thereof according to claim 17, wherein R¹⁴ and R¹⁵ are a sulfate group optionally substituted with one selected from the group consisting of sodium, potassium, copper, calcium, iron, manganese, zinc, ammonium, barium and lithium.

19. The reducing end glucuronic acid type oligochondroitin or a reducing end glucuronic acid type oligochondroitin sulfate, or the salt or derivative thereof according to claim 17, wherein n is 3 to 6.

20. A pharmaceutical composition comprising at least one selected from the group consisting of a reducing end glucuronic acid type oligochondroitin and a reducing end glucuronic acid type oligochondioitin sulfate, and the salt and derivative thereof according to claim 17 together with a pharmacologically acceptable carrier.

21. The pharmaceutical composition according to claim 20 for improving, treating or preventing a condition or diseases induced by a CD44 molecule.

22. The pharmaceutical composition according to claim 21 for treating an autoimmune disease, arthritis, an allergic disease or a cancer, or for modulating immunity or for inducing cell differentiation or cell apotosis.

23. Use of a reducing end glucuronic acid type oligochondroitin or a reducing end glucuronic acid type oligochondroitin sulfate, or a salt or derivative thereof according to claim 17 for the preparation of a pharmaceutical composition for improving, treating or preventing a condition or diseases induced by a CD44 molecule.

24. A method for improving, treating or preventing a condition or diseases induced by a CD44 molecule which comprise administering a reducing end glucuronic acid type oligochondroitin or a reducing end glucuronic acid type oligochondroitin sulfate, or a salt or derivative thereof according to claim 17.
